(19) United States
(12) Patent Application Publication
(10) Pub. No.: US 2015/0037793 A1
(43) Pub. Date: Feb. 5, 2015

(54) DETECTION METHODS FOR OIL PALM SHELL ALLELES

(71) Applicant: Malaysian Palm Oil Board, KAJANG (MY)

(72) Inventors: Rajinder SINGH, Kuala Lumpur (MY); Leslie Low Eng Ti, Kuala Lumpur (MY); Leslie Ooi Cheng Li, Kuala Lumpur (MY); Meilina Ong Abdullah, Seremban (MY); Rajanaidu Nookiah, Kuala Lumpur (MY); Ravigadevi Sambanthamurthi, Selangor (MY); Jared Ordway, St. Louis, MO (US); Nathan D. Lakey, Chesterfield, MO (US); Steven W. Smith, Fitchburg, WI (US); Rob Martienssen, Cold Spring Harbor, NY (US); Michael Hogan, Ballwin, MO (US)

(21) Appl. No.: 14/334,825

(22) Filed: Jul. 18, 2014

Related U.S. Application Data
(60) Provisional application No. 61/847,853, filed on Jul. 18, 2013.

(51) Int. Cl. C12Q 1/68

(52) U.S. Cl. CPC C12Q 1/6895; C12Q 2600/13; C12Q 2600/158
USPC 435/6.11

(57) ABSTRACT

Methods, compositions, and kits for determining SHELL genotype and predicting shell fruit form of oil palm plants.

### CROSS-REFERENCE TO RELATED PATENT APPLICATIONS

The present application claims the benefit of priority to U.S. Provisional Patent Application No. 61/847,853, filed on Jul. 18, 2013, the contents of which are hereby incorporated by reference in their entirety and for all purposes.

### BACKGROUND OF THE INVENTION

The oil palm (E. guineensis, E. oleifera, and hybrids thereof) can be classified into separate groups based on its fruit characteristics, and has three naturally occurring fruit forms which vary in shell thickness and oil yield. Dura type palms are homozygous for a wild type allele of the SHELL gene (Sh⁺/Sh⁺), have a thick seed coat or shell (2-8 mm) and produce approximately 5.3 tons of oil per hectare per year. Tenera type palms are heterozygous for a wild type and mutant allele of the SHELL gene (Sh⁺/sh⁻), have a relatively thin shell surrounded by a distinct fiber ring, and produce approximately 7.4 tons of oil per hectare per year. Finally pisifera type palms are homozygous for a mutant allele of the SHELL gene (sh⁻/sh⁻), have no seed coat or shell, and are usually female sterile (Hartley, C. W. S. 1988. The botany of oil palm. In The oil palm (3rd edition), pp: 47-94, Longman, London). Therefore the inheritance of the single gene controlling fruit shell phenotype is a major contributor to palm oil yield.

Tenera palms are simply hybrids between the dura and pisifera palms. Whitmore (Whitmore, T. C. 1973. The Palms of Malaya. Longmans, Malaysia, pp: 56-58) described the various fruit forms as different varieties of oil palm. However, Latiff (Latiff, A. 2000. The Biology of the Genus Elaeis. In: Advances in Oil Palm Research, Volume 1, ed. Y. Basiron, B. S. Jalani, and K. W. Chan, pp: 19-38, Malaysian Palm Oil Board (MPOB)) was in agreement with Purseglove (Purseglove, J. W. 1972. Tropical Crops. Monocotyledons. Longman, London. pp: 607) that varieties or cultivars as proposed by Whitmore (1973), do not occur in the strict sense in this species. As such, Latiff (2000) proposed the term “race” to differentiate dura, pisifera and tenera. Race was considered an appropriate term as it reflects a permanent microspecies, where the different races are capable of exchanging genes with one another, which has been adequately demonstrated in the different fruit forms observed in oil palm (Latiff, 2000). In fact, the characteristics of the three different races turn out to be controlled simply by the inheritance of a single gene. Genetic studies revealed that the SHELL gene shows co-dominant monogenic inheritance, which is exploitable in breeding programs (Beirnaert, A. and Vanderweyen, R. 1941. Contribution a l′etude genetique et biometrique des varieties d′Elaeis guineensis Jacq. Publs. INEAC, Series Ser. Sci. (27):101).

Tenera fruit forms have a higher mesocarp to fruit ratio than dura, which directly translates to significantly higher oil yield than either the dura or pisifera palm (as illustrated in Table 1). The pisifera palm is usually female sterile and does not produce fruit, and the fruit bunches, if produced, rot prematurely.

TABLE 1
Comparison of dura, tenera and pisifera fruit forms

Fruit Form

| Characteristic | Dura | Tenera | Pisifera* |
|---|---|---|---|
| Shell thickness (mm) | 2-8 | 0.5-3 | Absence of shell |
| Fibre Ring ** | Absent | Present | Absent |
| Mesocarp Content | 35-55 | 60-96 | 95 |
| (% fruit weight) | | | |
| Kernel Content | 7-20 | 3-15 | — |
| (% fruit weight) | | | |
| Oil to Bunch (%) | 16 | 26 | — |
| Oil Yield (t/ha/yr) | 5.3 | 7.4 | — |

*usually female sterile, bunches rot prematurely
** fibre ring is present in the mesocarp and often used as diagnostic tool to differentiate dura and tenera palms.
(Source: Hardon, J. J., Rao, V., and Rajanaidu, N. 1985. A review of oil palm breeding. In Progress in Plant Breeding, ed G. E. Rusell, pp 139-163, Butterworths, UK., 1985; Hartley, 1988)

Since the goal of the breeding programs in oil palm is to produce planting materials with higher oil yield, the tenera palm is the preferred choice for commercial planting. It is for this reason that substantial resources are invested by commercial seed producers to cross selected dura and pisifera palms in hybrid seed production. And despite the many advances which have been made in the production of hybrid oil palm seeds, two significant problems remain in the seed production process. First, batches of tenera seeds, which will produce the high oil yield tenera type palm, are often contaminated with dura seeds (Donough, C. R. and Law, I. H. 1995. Breeding and selection for seed production at Pamol Plantations Sdn Bhd and early performance of Pamol D x P. Planter 71:513-530). Today, it is estimated that dura contamination of tenera seeds can reach rates of approximately 5% (reduced from as high as 20-30% in the early 1990's as the result of improved quality control practices). Seed contamination is due in part to the difficulties of producing pure tenera seeds in open plantation conditions, where workers use ladders to manually pollinate tall trees, and where palm flowers for a given bunch mature over a period time, making it difficult to pollinate all flowers in a bunch with a single manual pollination event. Some flowers of the bunch may have matured prior to manual pollination and therefore may have had the opportunity to be wind pollinated from an unknown tree, thereby producing contaminant seeds in the bunch. Alternatively premature flowers may exist in the bunch at the time of manual pollination, and may mature after the pollination occurred allowing them to be wind pollinated from an unknown tree thereby producing contaminant seeds in the bunch.

Identification of the fruit type of a given seed, or of a given plant arising from a given seed is typically performed after the plant has matured enough to produce a first batch of fruit, which typically takes approximately six years after germination. Notably, in the six year interval from germination to fruit production, significant land, labor, financial and energy resources are invested into what are believed to be tenera trees, some of which will ultimately be of the unwanted low yielding contaminant fruit types. By the time these suboptimal trees are identified, it is impractical to remove them from the field and replace them with tenera trees, and thus growers achieve lower palm oil yields for the 25 to 30 year production life of the contaminant trees. Therefore, the issue of contamination of batches of tenera seeds with dura or pisifera seeds is a problem for oil palm breeding, underscoring the need for a method to predict the fruit type of seeds and nursery plantlets with high accuracy.

A second problem in the current seed production process is the investment seed producers make in maintaining dura and pisifera lines, and in the other expenses incurred in the hybrid seed production process. For example, to produce lines which maintain a pisifera allele, tenera palms are often selfed or crossed with another tenera palm. In this process, at least 25% of the progeny of such a cross are dura, based on Mendelian inheritance, and yet are cultivated in fields designated for pisifera maintenance for up to 6 years before they bear fruit and can be phenotyped.

### BRIEF SUMMARY OF THE INVENTION

In some embodiments, the present invention provides a method for predicting a shell fruit form of an oil palm seed or plant (e.g., dura, tenera, or pisifera) comprising amplifying DNA; digesting DNA comprising SEQ ID NO:4 from the seed or plant by contacting the DNA, or a portion thereof, with an endonuclease that distinguishes between SHELL genotypes; and determining the presence or absence of cleavage of the DNA by the endonuclease, thereby predicting the shell fruit form of the seed or plant.

In some cases, the method for predicting a shell fruit form further includes DNA amplification.

In some cases, the amplifying generates an amplicon and the digesting comprises digesting the amplicon with the endonuclease. In other cases, the digesting occurs before the amplifying. The amplifying can be amplification via polymerase chain reaction or isothermal amplification. In some cases, the amplification is linear amplification. In other cases, the amplification is exponential amplification. In some cases, the isothermal amplification is loop-mediated amplification (LAMP). In some cases, SHELL DNA is not amplified if cleaved, and amplified if uncleaved. In some cases, the amplifying is quantitative. In some cases, the amplification is real-time amplification.

In some cases, the endonuclease cleaves a nucleic acid encoding a wild-type SHELL allele, or a portion thereof but does not cleave a nucleic acid encoding a mutant SHELL allele, or a portion thereof. For example, the endonuclease cleaves a nucleic acid containing SEQ ID NO:1, but does not cleave a nucleic acid containing SEQ ID NOs:2 or 3. In other cases, the endonuclease cleaves a nucleic acid encoding a mutant SHELL allele, or a portion thereof but does not cleave a nucleic acid encoding a wild-type SHELL allele, or a portion thereof. For example, the endonuclease cleaves a nucleic acid containing SEQ ID NOs:2 or 3, but does not cleave a nucleic acid containing SEQ ID NO:1. The mutant SHELL allele can be an Sh^{MPOB} allele or an sh^{AVROS} allele. In some cases, the nucleic acid cleaved by the endonuclease is resistant to amplification. In some cases, a “portion thereof” can mean at least about 2, 3, 4, 5, 6, 7, 8, 10, 12, 15, 20, 25, 30, 35, 50, 100, 150, 200, 250, 500 or more continuous nucleotides of a SHELL gene.

In some cases, the endonuclease is Eco57I, or an isoschizomer thereof. In one aspect, Eco57I cleaves a nucleic acid encoding a wild-type SHELL allele, or a portion thereof, but does not cleave a nucleic acid encoding an sh^{MPOB} SHELL allele, or a portion thereof. For example, Eco57I can cleave a nucleic acid containing SEQ ID NO:1, but not cleave a nucleic acid containing SEQ ID NO:2. In some cases, a “portion thereof” can mean at least about 2, 3, 4, 5, 6, 7, 8, 10, 12, 15, 20, 25, 30, 35, 50, 100, 150, 200, 250, 500 or more continuous nucleotides of a SHELL gene.

In some cases, the endonuclease is HindIII, or an isoschizomer thereof. In one aspect, HindIII cleaves a nucleic acid encoding a wild-type SHELL allele, or a portion thereof but does not cleave a nucleic acid encoding an sh^{AVROS} SHELL allele, or a portion thereof. For example, HindIII cleaves a nucleic acid containing SEQ ID NO:1, but does not cleave a nucleic acid containing SEQ ID NO:3. In some cases, a “portion thereof” can mean at least about 2, 3, 4, 5, 6, 7, 8, 10, 12, 15, 20, 25, 30, 35, 50, 100, 150, 200, 250, 500 or more continuous nucleotides of a SHELL gene.

In some cases, the DNA, or a portion thereof, is contacted with a second endonuclease, such as HindIII or Eco57I. For example, a portion of the nucleic acid is digested with the first endonuclease and cleavage of the nucleic acid by the first endonuclease is detected, and a portion of the nucleic acid is separately digested with the second endonuclease and cleavage of the nucleic acid by the second endonuclease is detected.

In some cases, the second endonuclease distinguishes between SHELL genotypes. For example, the second endonuclease cleaves a nucleic acid encoding a wild-type SHELL allele, or a portion thereof, but does not cleave a nucleic acid encoding a mutant SHELL allele, or a portion thereof. For example, the second endonuclease cleaves a nucleic acid containing SEQ ID NO:1, but does not cleave a nucleic acid containing SEQ ID NOs:2 or 3. In other cases, the second endonuclease cleaves a nucleic acid encoding a mutant SHELL allele, or a portion thereof but does not cleave a nucleic acid encoding a wild-type SHELL allele, or a portion thereof. For example, the endonuclease cleaves a nucleic acid containing SEQ ID NOs:2 or 3, but does not cleave a nucleic acid containing SEQ ID NO:1. The mutant SHELL allele can be an Sh^{MPOB} allele or an sh^{AVROS} allele. In some cases, the nucleic acid cleaved by the second endonuclease is resistant to amplification. In some cases, a “portion thereof” can mean at least about 2, 3, 4, 5, 6, 7, 8, 10, 12, 15, 20, 25, 30, 35, 50, 100, 150, 200, 250, 500, 700, 750, 1000, 1500, 2000, 2500, 5000 or more continuous nucleotides of a SHELL gene.

In some cases, the method further comprises sorting the seed or plant on the basis of the predicted shell fruit form. The seed or plant can be sorted between dura, tenera, and pisifera fruit forms. The sorting can comprise selecting the seed or plant for cultivation or breeding on the basis of the predicted shell fruit form.

In another embodiment, the present invention provides a kit comprising: an oligonucleotide primer that primes the amplification of a nucleic acid comprising SEQ ID NO:4; and an endonuclease that distinguishes between SHELL genotypes. In some cases, the oligonucleotide primer comprises SEQ ID NO:4 or a reverse complement thereof. In some cases, the oligonucleotide primer comprises or consists of SEQ ID NOs: 9 or 10 or a reverse complement thereof.

The kit can further comprise a second oligonucleotide primer that hybridizes to an oil palm plant genome within about 8, 10, 15, 30, 50, 75, 100, 125, 150, 200, 300, 500, 750, 1000, or 1500 bp, or about 2, 2.5, 3, 5, 7.5, or 10 kb of the first oligonucleotide primer. The second and first primer can flank at least about 8, 10, 15, 30, 50, 75, 100, 125, 150, 200, 300, 500, 750, 1000, or 1500 bp, or about 2, 2.5, 3, 5, 7.5, or 10 kb of continuous nucleotides containing the SHELL gene. In some cases, the second primer comprises or consists of SEQ ID NOs:9, or 10 or a reverse complement thereof.

In some cases, the endonuclease cleaves a nucleic acid encoding a wild-type SHELL allele, or a portion thereof, such as a nucleic acid sequence containing SEQ ID NO:1, but does not cleave a nucleic acid encoding a mutant SHELL allele, or a portion thereof, such as a nucleic acid sequence containing SEQ ID NOs:2 or 3. In other cases, the endonuclease cleaves a nucleic acid encoding a mutant SHELL allele, or a portion thereof, (e.g., a nucleic acid sequence containing SEQ ID NOs:2 or 3) but does not cleave a nucleic acid encoding a wild-type SHELL allele, or a portion thereof, (e.g., a nucleic acid sequence containing SEQ ID NO:1). The mutant SHELL allele can be selected from the group consisting of an sh^{MPOB} allele and an sh^{AVROS} allele. In some cases, the endonuclease is Eco57I, AcuI, or an isoschizomer thereof. In some cases, a “portion thereof” can mean at least about 2, 3, 4, 5, 6, 7, 8, 10, 12, 15, 20, 25, 30, 35, 50, 100, 150, 200, 250, 500 or more continuous nucleotides of a SHELL gene.

In some cases, the kit further comprises a second endonuclease. The second endonuclease can be HindIII or an isoschizomer thereof.

In some cases, the kit can further comprise a control oligonucleotide, polynucleotide, or DNA sample. The control oligonucleotide, oligonucleotide, polynucleotide, or DNA sample can contain nucleic acid encoding a Sh^{DeliDura}, sh^{MPOB}, or sh^{AVROS} allele or a portion thereof.

### DEFINITIONS

As used herein, the terms “nucleic acid,” “polynucleotide” and “oligonucleotide” refer to nucleic acid regions, nucleic acid segments, nucleic acid sequences, primers, probes, amplicons and oligomer fragments. The terms are not limited by length and are generic to linear polymers of polydeoxyribonucleotides (containing 2-deoxy-D-ribose), polyribonucleotides (containing D-ribose), and any other N-glycoside of a purine or pyrimidine base, or modified purine or pyrimidine bases. These terms include double- and single-stranded DNA, as well as double- and single-stranded RNA. A nucleic acid, polynucleotide or oligonucleotide can include genomic DNA, cDNA, RNA, tRNA, or rRNA. The nucleic acid, polynucleotide or oligonucleotide can be labeled or unlabeled.

A nucleic acid, polynucleotide or oligonucleotide can comprise, for example, phosphodiester linkages or modified linkages including, but not limited to phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages.

A nucleic acid, polynucleotide or oligonucleotide can comprise the five biologically occurring bases (adenine, guanine, thymine, cytosine and uracil) and/or bases other than the five biologically occurring bases.

The terms “label” and “detectable label” interchangeably refer to a composition detectable by spectroscopic, photochemical, biochemical, immunochemical, chemical, or other physical means. For example, useful labels include fluorescent dyes, luminescent agents, radioisotopes (e.g., ³²P, ³H), electron-dense reagents, enzymes, biotin, digoxigenin, or haptens and proteins, nucleic acids, or other entities which can be made detectable, (e.g., by incorporating a radiolabel into an oligonucleotide, peptide, or antibody specifically reactive with a target molecule). Any method known in the art for conjugating, e.g., for conjugating a probe to a label, can be employed, e.g., using methods described in Hermanson, Bioconjugate Techniques 1996, Academic Press, Inc., San Diego.

A molecule that is “linked” or “conjugated” to a label (e.g., as for a labeled probe as described herein) is one that is bound, either covalently, through a linker or a chemical bond, or noncovalently, through ionic, van der Waals, electrostatic, or hydrogen bonds to a label such that the presence of the molecule can be detected by detecting the presence of the label bound to the molecule.

Optimal alignment of sequences for comparison may be conducted by the local homology algorithm of Smith and Waterman Add. APL. Math. 2:482 (1981), by the homology alignment algorithm of Needle man and Wunsch J. Mol. Biol. 48:443 (1970), by the search for similarity method of Pearson and Lipman Proc. Nati. Acad. Sci. (U.S.A.) 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polypeptide sequences means that a polypeptide comprises a sequence that has at least 75% sequence identity. Alternatively, percent identity can be any integer from 75% to 100%. Exemplary embodiments include at least: 75%, 80%, 85%, 90%, 95%, or 99% compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like. Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

Another indication that nucleotide sequences are substantially identical is if two molecules hybridize to each other, or a third nucleic acid, under stringent conditions. Stringent conditions are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is about 0.02 molar at pH 7 and the temperature is at least about 60° C.

As used herein, the term “Sh^{DeliDura}” refers to the wild-type allele (Sh⁺) of the oil palm SHELL gene. When present as a homozygous allele, Sh^{DeliDura} plants are generally of the dura fruit form phenotype. The nucleic acid sequence of the region of Sh^{DeliDura} that is polymorphic with respect to the other naturally occurring SHELL alleles is provided by SEQ ID NO:1. Similarly, “sh^{MPOB}” refers to a naturally occurring mutant SHELL allele (sh⁻) that can confer a tenera or pisifera phenotype as described herein. The nucleic acid sequence of sh^{MPOB} that is polymorphic with respect to the other naturally occurring SHELL alleles is provided by SEQ ID NO:2. Similarly, “sh^{AVROS}” refers to a naturally occurring mutant SHELL allele (sh⁻) that can confer a tenera or pisifera phenotype as described herein. The nucleic acid sequence of sh^{AVROS} that is polymorphic with respect to the other naturally occurring SHELL alleles is provided by SEQ ID NO:3. A consensus sequence of the polymorphic region of the Sh^{DeliDura}, sh^{MPOB}, and sh^{AVROS} SHELL alleles is also provided herein as SEQ ID NO:4.

Thus, SEQ ID NO:1 contains an Eco57I endonuclease recognition site and a HindIII endonuclease recognition site. In contrast, SEQ ID NO:2 contains a HindIII recognition site but no Eco57I recognition site. Similarly, SEQ ID NO:3 contains an Eco57I recognition site but no HindIII recognition site.

The full length SHELL nucleotide cDNA sequences for the wild-type, MPOB, and AVROS alleles are provided by SEQ ID NOs: 5-7 respectively. SEQ ID NO: 8 is an approximately 27 kb genomic interval of the oil palm plant genome containing the approximately 22 kb SHELL gene and approximately 5 kb of genomic sequence upstream of the SHELL gene.

The sequences provided in SEQ ID NOs:1-7 are representative sequences and different individual palm plants can have a nucleic acid sequence having one, two, three, or more nucleic acid substitutions, additions, or deletions relative to SEQ ID NOs: 1-7 due, for example, to natural variation. Similarly, SEQ ID NO:8 is a representative sequence and different individual palm plants can have a nucleic acid sequence having one, two, three, four, five, six, seven, eight, nine, ten, eleven, twelve, or more nucleic acid substitutions, additions, or deletions relative to SEQ ID NO: 8 due, for example, to natural variation.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous. The class of plants also includes plants of the genus Elaeis such as E. guineensis and E. oleifera and hybrids thereof.

### BRIEF DESCRIPTION OF THE DRAWINGS

FIG. 1. Illustrates a detection assay for determining SHELL genotype and predicting shell fruit form. A. The wild-type SHELL allele Sh^{DeliDura} has both an intact Eco57I/AcuI recognition site and an intact HindIII recognition site. B. The mutant SHELL allele sh^{MPOB} has an intact HindIII site, but the Eco57I/AcuI recognition site is absent due to a “T” (sh^{DeliDura}) to “C” (sh^{MPOB}) base change in the site, as marked by an arrow. C. The mutant SHELL allele sh^{AVROS} has an intact Eco57I/AcuI recognition site, but the HindIII site is absent due to an “A” (sh^{DeliDura}) to “T” (sh^{AVROS}) base change in the site, as marked by an arrow.

FIG. 2. A. Gel electrophoretic migration patterns as measured on an Agilent Bioanalyzer LabChip (P/N: G2938-90015) for all possible restriction fragments after digestion with no enzyme, Eco57I/AcuI, and HindIII, of ˜350 bp SHELL amplicons of Sh^{DeliDura}, sh^{MPOB}, and sh^{AVROS} Peaks corresponding to the upper (1,500 bp) and lower (15 bp) size standard markers flank the three experimental fragment peaks. A single peak corresponding to uncut amplicon (˜350 bp), and peaks corresponding to the two restriction products of either HindIII or Eco57I/AcuI digestion (˜100 bp and ˜250 bp) are also visible.

B. Reaction products of DNA from dura palm samples yielded a ˜350 bp band in the ‘No enzyme’ lane, and a ˜250 bp and ˜100 bp band in each of the ‘ AcuI’ and ‘HindIII’ lanes.

C. Reaction products of DNA from tenera palm samples of the sh^{MPOB}/sh^{DeliDura} genotype yielded a ˜350 bp band in each of the ‘No enzyme’ and ‘ AcuI’ lanes, and a ˜250 bp and ˜100 bp band in each of the ‘ AcuI’ and ‘HindIII’ lanes.

D. Reaction products of DNA from tenera palm samples of the sh^{AVROS}/sh^{DeliDura} genotype yielded a ˜350 bp band in each of the ‘No enzyme’ and ‘HindIII’ lanes, and a ˜250 bp and ˜100 bp band in each of the ‘ Acu I’ and ‘Hind III’ lanes.

E. Reaction products of DNA from pisifera palm samples that are homozygous for the sh^{MPOB} allele (sh^{MPOB}/sh^{MPOB}) yielded a ˜350 bp band in each of the ‘No enzyme’ and ‘ Acu I’ lanes, and a ˜250 bp and ˜100 bp band in the ‘Hind III’ lane.

F. Reaction products of DNA from pisifera palm samples that are homozygous for the sh^{AVROS} allele (sh^{AVROS}/sh^{AVROS}) yielded a ˜350 bp band in each of the ‘No enzyme’ and ‘Hind III’ lanes, and a ˜250 bp and ˜100 bp band in the “Acu I” lane.

G. Reaction products of DNA from pisifera palm samples that are heterozygous sh^{MPOB}/sh^{AVROS} yield a ˜350 bp band in all three lanes and two bands of ˜250 bp and ˜100 bp in both the Acu I and ‘ Hind III’ lanes.

FIG. 3. Depicts a longitudinal cross section of an oil palm seed, passing through the embryo and the germ pore containing the fibre plug which is adjacent to the embryo. Once the mesocarp tissue (a fleshy oily fruit layer) has been removed, a small 2-3 cm seed can been seen, weighing 1 to 13 grams (4 grams on average) and having a fibrous ‘coconut-like’ shell. A. The shell layer is fibrous and maternally derived, and thickness of the shell is determined by the SHELL gene genotype of the mother palm, and not on the genotype of the newly fertilized embryo. B. The large endosperm, also referred to as the kernel, is a triploid tissue (i.e., contains three independent sets of chromosomes) with two identical maternal chromosome sets (derived from the same gametophyte as the single maternal chromosome set present in the embryo), and one paternal chromosome set (also identical to the paternal chromosome set present in the embryo). C. The small embryo, around 3 mm in length, is positioned near the base of the seed and adjacent to one of three germ pores containing a fibre plug D. which is shed as the embryo grows and emerges from the oil palm seed. The nuclear genomes of the embryo and the endosperm are identical, except the endosperm has 2 sets of identical maternal chromosomes maternal, and one set of paternal chromosomes, while the embryo has one set of paternal and maternal chromosomes.

FIG. 4. Depicts a longitudinal cross section of two oil palm seeds oriented in the same direction. The section passes through the embryo and germ pore containing the fibre plug which is adjacent to the embryo. A. The portion of the seed opposite the three germ pores does not contain the embryo. Sampling endosperm material from this zone will not result in wounding or killing the developing embryo. B. The portion of the seed adjacent to the three germ pores contains the embryo. Sampling endosperm material from this zone may result in wounding or killing the developing embryo.

### DETAILED DESCRIPTION OF THE INVENTION

### I. Introduction

Described herein are methods, compositions, and kits for predicting the shell fruit form (e.g., dura, tenera, or pisifera) of an oil palm plant. Typically, shell fruit form is determined by the presence or absence of three different naturally occurring SHELL alleles, Sh^{DeliDura} (which is wild-type), and sh^{MPOB}, and sh^{AVROS} (which are mutant alleles). Moreover the SHELL locus exhibits co-dominance. Thus, oil palm shell fruit forms follow the following pattern:
plants with a dura phenotype possess two copies of the wild-type Sh^{DeliDura} allele;
plants with a tenera phenotype possess one copy of the wild-type SHELL allele, and either one copy of the sh^{MPOB} allele or one copy of the sh^{AVROS} allele; and
plants with a pisifera phenotype possess either two copies of the sh^{MPOB} or sh^{AVROS} alleles or one copy each of the sh^{MPOB} and sh^{AVROS} alleles.
   Therefore, the shell fruit form of a plant can be accurately predicted by assaying for the presence of the three naturally occurring SHELL alleles (Sh^{DeliDura}, sh^{MPOB}, and sh^{AVROS}).

Moreover, the inventors have discovered that the three naturally occurring SHELL alleles can be differentially detected using, for example, restriction enzyme digestion and/or nucleic acid amplification (e.g., PCR). For example, the restriction endonuclease Eco57I or AcuI or an isoschizomer thereof can be contacted with, optionally amplified, nucleic acid containing the SHELL locus, and optionally amplified. Eco57I or AcuI will cleave nucleic acid encoding SHELL that contains the Sh^{DeliDura} and sh^{AVROS} alleles, but not the sh^{MPOB} allele. Similarly, the restriction endonuclease HindIII or an isoschizomer thereof, cleaves nucleic acid encoding SHELL that contains the Sh^{DeliDura} and sh^{MPOB} alleles, but not sh^{AVROS} allele. Cleavage can then be detected using a variety of techniques, including but not limited to amplification and/or electrophoresis. The resulting HindIII and Eco57I or AcuI SHELL allele cleavage patterns are unique for each of the six naturally occurring genotypes as described herein. Thus, the SHELL genotype can be determined for any given plant and the shell fruit form thereby predicted.

Moreover, any reagent or set of reagents that can distinguish between the three naturally occurring SHELL alleles can be used to predict the shell fruit form. Such reagents include, but are not limited to, one or more endonucleases, catalytic nucleic acids (e.g., ribozymes) that cleave nucleic acid substrates (e.g., one or more SHELL alleles, or portions thereof) in a sequence dependent manner, nucleic acid binding proteins that bind to one or more SHELL alleles, or portions thereof, in a sequence dependent manner, or oligonucleotides that hybridize to and/or prime polymerization or amplification of one or more SHELL alleles, or portions thereof, in a sequence dependent manner.

Also described herein are methods of sorting or selecting seeds or plants based on predicted shell fruit form. Methods, compositions, and kits for predicting shell fruit form or sorting or selecting plants or seeds based on the predicted shell fruit form can be useful for oil palm plant cultivators and breeders by reducing the typical six year period required to determine shell fruit form using traditional methods, and by increasing the accuracy of fruit form predictions.

The ability to identify and separate out the different fruit forms greatly improves management practice, as the different fruit forms can be planted separately in the field. For example, pisifera trees can be identified and planted in high density to encourage optimal male flower formation and increased pollen production. It is known that male inflorescence development is increased in pisifera palms when planted in pure plots at high density. It follows then that increased pollen production of high density pure pisifera plots would increase seed set in neighboring dura palms, which in turn would boost overall yield in the production of hybrid tenera seed. In yet another example, tenera palms which need to be evaluated for performance can likewise be planted separately and away from contaminant pisifera palms. Pisifera palms exhibit more vigorous vegetative growth than dura and tenera palms, and when planted in proximity of palms which are undergoing trait evaluation, compete for resources and mask the performance of neighboring palms. Therefore, an accurate test that can identify and segregate palms into different fruit forms at the seed or seedling stage, enables growers to intentionally plant given fruit forms separately in fields for various purposes, thereby greatly improving management practice.

### II. Compositions

### A. Proteins

Reagents are described herein that distinguish between SHELL genotypes, e.g., by recognizing a nucleic acid sequence that is indicative of a SHELL genotype. In some embodiments, the recognition sequence lies within the SHELL gene. For example, the reagent can beEco57I or an isoschizomer thereof which cleaves an Eco57I recognition site that is present in the Sh^{DeliDura} and sh^{AVROS} alleles, but not in the sh^{MPOB} allele. As another example, the reagent can be HindIII or an isoschizomer thereof which cleaves a HindIII recognition site that is present in the Sh^{DeliDura} and sh^{MPOB} alleles, but not in the sh^{AVROS} allele.

In one embodiment, the reagent that distinguishes between SHELL genotypes is an endonuclease that is specific for the Sh^{DeliDura} and sh^{AVROS} alleles. In some cases, the endonuclease can recognize Sh^{DeliDura} and sh^{AVROS} sequences, but not an sh^{MPOB} sequence. For example, Eco57I or AcuI cleaves Sh^{DeliDura} and sh^{AVROS} sequences (e.g., nucleic acids containing SEQ ID NOs:1 and 3 respectively), but not an sh^{MPOB} sequence (e.g., a nucleic acid containing SEQ ID NO:2). In another embodiment, the endonuclease can be specific for the Sh^{DeliDura} and sh^{MPOB} alleles. In some cases, the endonuclease can recognize Sh^{DeliDura} and Sh^{MPOB} sequences, but not an sh^{AVROS} sequence. For example, HindIII cleaves Sh^{DeliDura} and Sh^{MPOB} sequences (e.g., nucleic acids containing SEQ ID NOs:1 and 2 respectively), but not an sh^{AVROS} sequence (e.g., nucleic acid containing SEQ ID NO:3). Thus, the SHELL genotype can be determined and the shell fruit form predicted by contacting oil palm nucleic acid with the endonuclease and detecting whether the protein has recognized (e.g., cleaved) the SHELL locus. In some cases, the detecting is quantitative such that recognition of one or both copies of the SHELL locus can be distinguished. In some cases, cleavage by a restriction endonuclease will block subsequent amplification of the sequence, for example by cleaving the target sequence between a primer pair. In this case, lack of amplification (assuming appropriate controls) indicates cleavage of the restriction site.

In other embodiments, the reagent is a protein that is specific for the wild-type SHELL allele but not for one or more mutant SHELL alleles. For example, a protein can recognize (e.g., bind to or cleave) a sequence present in the Sh^{DeliDura} allele that is not present in the sh^{MPOB} allele. As another example, a protein can recognize (e.g., bind to or cleave) a sequence present in the Sh^{DeliDura} allele that is not present in the sh^{AVROS} allele. As yet another example, a protein can recognize (e.g., bind to or cleave) a sequence present in the Sh^{DeliDura} allele that is not present in either the sh^{MPOB} allele or the sh^{AVROS} allele. Thus, the SHELL genotype can be determined and the shell fruit form predicted by contacting oil palm nucleic acid with the protein and detecting whether the protein has recognized (e.g., bound or cleaved) the SHELL locus. In some cases, the detecting is quantitative such that recognition of one or both copies of the SHELL locus can be distinguished. In some cases, the protein is an endonuclease and recognition is detected by detecting cleavage of the nucleic acid. Alternatively, the protein is a nucleic acid binding protein and recognition is detected by detecting the presence of the protein bound to the nucleic acid.

In some embodiments, the reagents that distinguish between SHELL genotypes are proteins that are specific for one or more mutant SHELL alleles. For example, the protein can recognize a sequence present in the sh^{MPOB} allele that is not present in the Sh^{DeliDura} allele. As another example, the protein can recognize a sequence present in the sh^{AVROS} allele that is not present in the Sh^{DeliDura} allele. As yet another example, the protein can recognize a sequence present in the sh^{MPOB} allele and the sh^{AVROS} allele that is not present in the Sh^{DeliDura} allele. Thus, the SHELL genotype can be determined and the shell fruit form predicted by contacting oil palm nucleic acid with the protein and detecting whether the protein has recognized the SHELL locus. In some cases, the detecting is quantitative such that recognition of one or both copies of the SHELL locus can be distinguished. In some cases, the protein is an endonuclease and recognition is detected by detecting cleavage of the nucleic acid. Alternatively, the protein is a nucleic acid binding protein and recognition is detected by detecting the presence of the protein bound to the nucleic acid.

In yet other embodiments, the protein can be specific for the sh^{AVROS} allele. For example, the protein can recognize a sh^{AVROS} sequence, but not a Sh^{DeliDura} or sh^{MPOB} sequence. Alternatively, the protein can be specific for the sh^{MPOB} allele. For example, the protein can recognize a sh^{MPOB} sequence, but not a Sh^{DeliDura} or sh^{AVROS} sequence. Thus, the SHELL genotype can be determined and the shell fruit form predicted by contacting oil palm nucleic acid with the protein and detecting whether the protein has recognized the SHELL locus. In some cases, the detecting is quantitative such that recognition of one or both copies of the SHELL locus can be distinguished. In some cases, the protein is an endonuclease and recognition is detected by detecting cleavage of the nucleic acid. Alternatively, the protein is a nucleic acid binding protein and recognition is detected by detecting the presence of the protein bound to the nucleic acid.

In some cases instead of recognizing a polymorphism within the SHELL gene, the protein recognizes a polymorphism (e.g., an SNP, RFLP, or other polymorphism) that is genetically linked to the SHELL locus. Thus, the protein can be used to infer the SHELL genotype of a child plant by tracking parental contribution of the polymorphism to the child. In some cases, the polymorphism and the SHELL locus are in close physical proximity on the oil palm plant genome (e.g., less than 10, 5, 4, 3, 2, 1, 0.1, or 0.01 cM, or less than 200, 100, 50, 50 or 10 kb). In such cases, the probability that the linked polymorphism and the SHELL allele of the parent will co-segregate is high. Thus, the inherited SHELL genotype can be inferred, and the shell fruit form thereby predicted with a high degree of confidence.

Exemplary proteins capable of distinguishing alleles can include any protein that distinguishes between nucleic acid sequences, e.g., transcription factors, bZIP proteins, HMG-box proteins, zinc-finger proteins, TALEs, TALENS, endonucleases, meganucleases, homing endonucleases, antibodies, and restriction endonucleases. In some cases, the protein is a nucleic acid binding protein (e.g., a transcription factor, zinc-finger protein, HMG-box protein, TALE, or bZIP protein) and recognition is detected by detecting the presence of the protein bound to the nucleic acid. In some cases, the nucleic acid is bound or immobilized to a solid support such as a planar substrate, a membrane, an array, or a bead. In some cases, the use of immobilized DNA facilitates the washing away of unbound detection reagent.

### B. Oligonucleotides

In some embodiments, the reagents that distinguish between SHELL genotypes are oligonucleotides (rather than proteins as described above) that are specific for one or more SHELL alleles, or specific for a polymorphism that is linked to one or more SHELL alleles. In some cases, the oligonucleotide is a catalytic nucleic acid (e.g., ribozyme), or a component of a catalytic nucleic acid that specifically cleaves one or more SHELL alleles in a sequence dependent manner. Detection of the sequence dependent cleavage can indicate the genotype and thus predict the phenotype of an oil palm plant. In other cases, the oligonucleotide hybridizes to one or more SHELL alleles in a sequence dependent manner and detection of hybridization can indicate the genotype and thus predict the phenotype of an oil palm plant. In still other cases, the oligonucleotide, or set of oligonucleotides, primes polymerization and/or amplification of one or more SHELL alleles in a sequence dependent manner and detection of polymerization or amplification can indicate genotype and thus predict the phenotype of an oil palm plant. An oligonucleotide, or set of oligonucleotides, can also be used in conjunction with one or more other detection reagents (e.g., proteins or nucleic acids) to detect binding or cleavage of a detection reagent to one or more SHELL alleles, for example by amplification of the SHELL locus or a portion thereof.

In some embodiments, the oligonucleotides specifically hybridize to one or more SHELL alleles. For example, the oligonucleotide can hybridize to a Sh^{DeliDura} sequence but not to a sh^{MPOB} sequence. As another example, the oligonucleotide can hybridize to a Sh^{DeliDura} sequence but not to a sh^{AVROS} sequence. As yet another example, the oligonucleotide can hybridize to the Sh^{DeliDura} sequence, but not to either the sh^{MPOB} or the sh^{AVROS} sequences. Thus, the SHELL genotype can be determined and the shell fruit form predicted by contacting oil palm nucleic acid with an oligonucleotide and detecting hybridization. In some cases, the detecting is quantitative such that hybridization to one or both copies of the SHELL locus can be distinguished.

In some cases, the oligonucleotides can selectively prime polymerization of a wild-type SHELL sequence but not one or more mutant SHELL sequences. For example, the oligonucleotide can prime polymerization of a Sh^{DeliDura} sequence but not a sh^{MPOB} sequence. As another example, the oligonucleotide can prime polymerization of a Sh^{DeliDura} sequence but not a sh^{AVROS} sequence. As yet another example, the oligonucleotide can prime polymerization of the Sh^{DeliDura} sequence, but not to either the sh^{MPOB} or the sh^{AVROS} sequences. Thus, the SHELL genotype can be determined and the shell fruit form predicted by contacting oil palm nucleic acid with an oligonucleotide, polymerizing, and detecting polymerization. In some cases, the detecting is quantitative such that polymerization from one or both copies of the SHELL locus can be distinguished.

In some embodiments, the reagents that distinguish between SHELL genotypes are oligonucleotides that are specific for one or more mutant SHELL alleles. For example, the oligonucleotide can hybridize to a sh^{MPOB} sequence but not to a Sh^{DeliDura} sequence. As another example, the oligonucleotide can hybridize to a sh^{AVROS} sequence but not to a Sh^{DeliDura} sequence. As yet another example, the oligonucleotide can hybridize to sh^{MPOB} and sh^{AVROS} sequences, but not to the Sh^{DeliDura} sequence. Thus, the SHELL genotype can be determined and the shell fruit form predicted by contacting oil palm nucleic acid with an oligonucleotide and detecting hybridization. In some cases, the detecting is quantitative such that hybridization to one or both copies of the SHELL locus can be distinguished.

In some cases, the oligonucleotides can selectively prime polymerization of one or more mutant SHELL alleles. For example, the oligonucleotide can prime polymerization of a sh^{MPOB} sequence but not a Sh^{DeliDura} sequence. As another example, the oligonucleotide can prime polymerization of a sh^{AVROS} sequence but not a Sh^{DeliDura} sequence. As yet another example, the oligonucleotide can prime polymerization of sh^{MPOB} and sh^{AVROS} sequences, but not the Sh^{DeliDura} sequence. Thus, the SHELL genotype can be determined and the shell fruit form predicted by contacting oil palm nucleic acid with an oligonucleotide, polymerizing, and detecting polymerization. In some cases, the detecting is quantitative such that polymerization from one or both copies of the SHELL locus can be distinguished.

In some embodiments, the reagents that distinguish between SHELL genotypes are oligonucleotides that are specific for Sh^{DeliDura} and sh^{AVROS} For example, the oligonucleotide can hybridize to Sh^{DeliDura} and sh^{AVROS} sequences, but not to the sh^{MPOB} sequence. In some cases, the oligonucleotide can prime polymerization of Sh^{DeliDura} and Sh^{AVROS} sequences, but not the sh^{MPOB} sequence. Thus, the SHELL genotype can be determined and the shell fruit form predicted by contacting oil palm nucleic acid with an oligonucleotide and detecting hybridization, or polymerizing, and detecting polymerization. In some cases, the detecting is quantitative such that hybridization or polymerization from one or both copies of the SHELL locus can be distinguished.

In some embodiments, the reagents that distinguish between SHELL genotypes are oligonucleotides that are specific for Sh^{DeliDura} and sh^{MPOB}. For example, the oligonucleotide can hybridize to Sh^{DeliDura} and sh^{MPOB} sequences, but not to the sh^{AVROS} sequence. In some cases, the oligonucleotide can prime polymerization of Sh^{DeliDura} and sh^{MPOB} sequences, but not the sh^{AVROS} sequence. Thus, the SHELL genotype can be determined and the shell fruit form predicted by contacting oil palm nucleic acid with an oligonucleotide and detecting hybridization, or polymerizing, and detecting polymerization. In some cases, the detecting is quantitative such that hybridization or polymerization from one or both copies of the SHELL locus can be distinguished.

In some embodiments, the reagents that distinguish between SHELL genotypes are oligonucleotides that are specific for sh^{AVROS} For example, the oligonucleotide can hybridize to a sh^{AVROS} sequence, but not to a Sh^{DeliDura} or sh^{MPOB} sequence. In some cases, the oligonucleotide can prime polymerization of an sh^{AVROS} sequence, but not a Sh^{DeliDura} or sh^{MPOB} sequence. Alternatively, the reagents that distinguish between SHELL genotypes are oligonucleotides that are specific for sh^{MPOB}. For example, the oligonucleotide can hybridize to a sh^{MPOB} sequence, but not to a Sh^{DeliDura} or sh^{AVROS} sequence. In some cases, the oligonucleotide can prime polymerization of a sh^{MPOB} sequence, but not an Sh^{DeliDura} or sh^{AVROS} sequence. Thus, the SHELL genotype can be determined and the shell fruit form predicted by contacting oil palm nucleic acid with an oligonucleotide and detecting hybridization, or polymerizing, and detecting polymerization. In some cases, the detecting is quantitative such that hybridization or polymerization from one or both copies of the SHELL locus can be distinguished.

In some cases, the oligonucleotide recognizes a polymorphism (e.g., an SNP, RFLP, or other polymorphism) that is genetically linked to the SHELL locus. Thus, the oligonucleotide can be used to infer the SHELL genotype of a child plant by tracking parental contribution of the polymorphism to the child. In some cases, the polymorphism and the SHELL locus are in close physical proximity on the oil palm plant genome (e.g., less than 10, 5, 4, 3, 2, 1, 0.1, or 0.01 cM). In such cases, the probability that the linked polymorphism and the SHELL allele of the parent will co-segregate is high. Thus, the inherited SHELL genotype can be inferred, and the shell fruit form thereby predicted with a high degree of confidence.

### II. Methods

### A. Detection

Described herein are methods for predicting the shell fruit form of an oil palm plant.

Exemplary methods include, but are not limited to contacting oil palm plant nucleic acid containing the SHELL gene with an endonuclease (e.g., Eco57I, AcuI, or an isoschizomer thereof) that cleaves Sh^{DeliDura} and sh^{AVROS} SHELL alleles, but does not cleave the sh^{MPOB} allele. Exemplary methods further include, but are not limited to contacting oil palm plant nucleic acid containing the SHELL gene with an endonuclease (e.g., HindIII or an isoschizomer thereof) that cleaves Sh^{DeliDura} and sh^{MPOB} SHELL alleles, but does not cleave the sh^{AVROS} allele. Exemplary methods also include contacting a portion of oil palm plant nucleic acid with a first endonuclease (e.g., Eco57I) and a portion of oil palm plant nucleic acid with a second endonuclease (e.g., HindIII). The resulting cleavage patterns can be analyzed to determine all six naturally occurring SHELL genotypes and thus predict all three naturally occurring shell fruit forms.

More generally, methods for predicting the shell fruit form of an oil palm plant include contacting nucleic acid containing the SHELL gene with a protein or oligonucleotide that recognizes the SHELL gene or a sequence linked to the SHELL gene and then detecting recognition (e.g., binding or cleavage). The detection reagent (e.g., protein or oligonucleotide) can be specific for one or more naturally occurring SHELL alleles (e.g., Sh^{DeliDura}, sh^{MPOB}, or sh^{AVROS}). In some cases, the method includes amplifying a SHELL gene sequence or a sequence linked to the SHELL gene and detecting the amplification. In some embodiments, the method includes a combination of contacting with a detection reagent and amplification. For example, the SHELL gene, or a portion thereof, can be amplified, and an oligonucleotide or protein detection reagent (e.g., a restriction enzyme such as Eco57I, AcuI, an isoschizomer thereof, HindIII or an isoschizomer thereof) can be contacted with the amplified nucleic acid. In some cases, further amplification can then be performed. Alternatively, the protein detection reagent can be contacted with nucleic acid and the SHELL gene, or a portion thereof, then amplified. In some embodiments, alleles, or portions thereof, that are recognized by the detection reagent (e.g., protein or oligonucleotide) are amplified. In other embodiments, alleles that are not recognized by the detection reagent, or portions thereof, are amplified and recognized alleles, or portions thereof, are not amplified.

In some embodiments, the methods include amplifying oil palm plant nucleic acid and contacting the amplified nucleic acid with a detection reagent (e.g., an oligonucleotide or a protein). The presence or activity of the detection reagent (e.g., binding or cleavage) can then be assayed as described herein. Alternatively, the nucleic acid can be contacted with the detection reagent, and then amplification can be performed. In some cases, SHELL alleles that are not recognized by the detection reagent can be amplified while SHELL alleles that are recognized by the detection reagent are not substantially amplified or are not amplified. In some cases, SHELL alleles that are recognized by the detection reagent can be amplified while SHELL alleles that are not recognized by the detection reagent are not substantially amplified or are not amplified.

Oil palm nucleic acid can be obtained from any suitable tissue of an oil palm plant. For example, oil palm nucleic acid can be obtained from a leaf, a stem, a root or a seed. In some cases, the oil palm nucleic acid is obtained from endosperm tissue of a seed. In some cases, the oil palm nucleic acid is obtained in such a manner that the oil palm plant or seed is not reduced in viability or is not substantially reduced in viability. For example, in some cases, sample extraction can reduce the number of viable plants or seeds in a population by less than about 20%, 15%, 10%, 5%, 2.5%, 1%, or less.

Samples can be extracted by grinding, cutting, slicing, piercing, needle coring, needle aspiration or the like. Sampling can be automated. For example, a machine can be used to take samples from a plant or seed, or to take samples from a plurality of plants or seeds. Sampling can also be performed manually.

In some cases, samples are purified prior to detection of SHELL genotype or prediction of fruit form phenotype. For example, samples can be centrifuged, extracted, or precipitated. Additional methods for purification of plant nucleic acids are known by those of skill in the art.

1. Endonuclease Detection

In some embodiments, contacting the oil palm nucleic acid (or an amplified portion thereof comprising at least a portion of the SHELL gene) with a detection reagent includes contacting the oil palm nucleic acid with an endonuclease that specifically recognizes one or more SHELL alleles under conditions that allow for sequence specific cleavage of the one or more recognized alleles. Such conditions will be dependent on the endonuclease employed, but generally include an aqueous buffer, salt (e.g., NaCl), and a divalent cation (e.g., Mg²⁺, Ca²⁺, etc.). The cleavage can be performed at any temperature at which the endonuclease is active, e.g., at least about 5, 7.5, 10, 15, 20, 25, 30, 35, 37, 40, 42, 45, 50, 55, or 65° C. The cleavage can be performed for any length of time such as about 1, 2, 3, 4, 5, 6, 7, 8, 9, 10, 12, 15, 17, 20, 25, 30, 35, 40, 45, 50, 60, 70, 90, 100, 120 minutes; about 2, 3, 4, 5, 6, 7, 8, 10, 12, 14, 16, 18, 20 hours, or about 1, 2, 3, or 4 days. In some cases, the oil palm nucleic acid or a portion thereof (e.g., the SHELL locus or a portion thereof) is amplified and then contacted with an endonuclease. Alternatively, the oil palm nucleic acid, or a portion thereof (e.g., the SHELL locus or a portion thereof) is contacted with an endonuclease and then amplified.

In some cases, cleavage of the nucleic acid prevents substantial amplification; therefore, lack of amplification indicates successful cleavage and thus presence of the allele or alleles recognized by the endonuclease detection reagent. For example, in some cases, amplification can require a primer pair and cleavage can disrupt the sequence of template nucleotides between the primer pair. Thus, in this case, a cleaved sequence will not be amplified, while the uncleaved sequence will be amplified. As another example, cleavage can disrupt a primer binding site thus preventing amplification of the cleaved sequence and allowing amplification of the uncleaved sequence.

Cleavage can be complete (e.g., all, substantially all, or greater than 50% of the SHELL locus is cleaved or cleavable) or partial (e.g., less than 50% of the SHELL locus is cleaved or cleavable). In some cases, complete cleavage can indicate the presence of a recognized SHELL allele and the absence of SHELL alleles that are not recognized. For example, complete cleavage can indicate that the plant is homozygous for an allele that is recognized by the detection reagent. Similarly, partial cleavage can indicate the presence of both a recognized SHELL allele and a SHELL allele that is not recognized. For example, partial cleavage can indicate heterozygosity at the SHELL locus.

In some embodiments, two or more endonucleases with differing specificities for one or more SHELL alleles are contacted with oil palm nucleic acid. In some cases, the oil palm nucleic acid is, optionally amplified, divided into separate reactions, optionally amplified, and each of the two or more endonucleases added to a separate reaction. One or more control reactions that include, e.g., no endonuclease, no nucleic acid, no amplification, or control sh^{DeliDura}, sh^{MPOB}, or sh^{AVROS} nucleic acid can also be included.

For example, an endonuclease that is specific for both the Sh^{DeliDura} allele and the sh^{AVROS} allele (e.g., Eco57I, AcuI, or an isoschizomer thereof) can be contacted with oil palm nucleic acid or a portion thereof (e.g., the SHELL locus or a portion thereof) in a first reaction, and an endonuclease specific for the Sh^{DeliDura} and sh^{MPOB} allele (e.g., HindIII or an isoschizomer thereof) can be contacted with oil palm nucleic acid or a portion thereof (e.g., the SHELL locus or a portion thereof) in a second reaction under conditions suitable for specific cleavage of the oil palm nucleic acid. The oil palm nucleic acid or a portion thereof (e.g., the SHELL locus or a portion thereof) can optionally then be amplified.

Cleavage can then be detected. Detection of complete cleavage in the first reaction indicates the presence of the Sh^{DeliDura} allele or the sh^{AVROS} allele. Detection of partial cleavage in the first reaction indicates the presence of the sh^{MPOB} allele and either the Sh^{DeliDura} allele or the sh^{AVROS} allele. Detection of no cleavage in the first reaction indicates the absence of the Sh^{DeliDura} allele and the sh^{AVROS} allele, thus inferring the presence of only the sh^{MPOB} allele and predicting a pisifera phenotype. Detection of complete cleavage in the second reaction indicates the presence of the Sh^{DeliDura} allele or the sh^{MPOB} allele. Detection of partial cleavage in the second reaction indicates the presence of the sh^{AVROS} allele and either the Sh^{DeliDura} allele or the sh^{MPOB} allele. Detection of no cleavage in the second portion indicates the absence of the Sh^{DeliDura} allele and the sh^{MPOB} allele, thus inferring the presence of only the sh^{AVROS} allele and predicting a pisifera phenotype.

Thus, the six most prevalent genotypes (Sh^{DeliDura}/sh^{DeliDura}, sh^{DeliDura}/sh^{MPOB}, Sh^{DeliDura}/sh^{AVROS}, sh^{MPOB}/sh^{MPOB}, sh^{MPOB}/sh^{AVROS} sh^{AVROS}/sh^{AVROS}) and their corresponding three fruit form phenotypes (dura, tenera, tenera, pisifera, pisifera, pisifera respectively) can be predicted based on comparing the cleavage pattern of the reaction containing the endonuclease that is specific for both the Sh^{DeliDura} allele and the sh^{AVROS} allele with the reaction containing the endonuclease specific for the Sh^{DeliDura} and sh^{MPOB} allele. Consequently, a dura phenotype (sh^{DeliDura}/sh^{DeliDura}) can be predicted by a cleavage pattern of complete cleavage in both reaction mixtures. Similarly, a tenera phenotype can be predicted by a cleavage pattern of partial cleavage in one reaction mixture and complete cleavage in the other. For example, Sh^{DeliDura}/sh^{MPOB} is indicated by partial cleavage in the first reaction mixture and complete cleavage in the second reaction mixture, thus predicting a tenera phenotype. Alternatively, Sh^{DeliDura}/sh^{AVROS} is indicated by complete cleavage in the first reaction mixture and partial cleavage in the second reaction mixture, thus predicting a tenera phenotype. Similarly, pisifera phenotypes can be predicted by no cleavage in any single reaction mixture or partial cleavage in both reaction mixtures.

In other embodiments, an endonuclease specific for the Sh^{DeliDura} allele can be contacted with oil palm nucleic acid, or a portion thereof (e.g., the SHELL locus or a portion thereof), under conditions suitable for specific cleavage of the oil palm nucleic acid. The oil palm nucleic acid or a portion thereof (e.g., the SHELL locus or a portion thereof) can optionally then be amplified. Cleavage can then be detected. Detection of complete cleavage can indicate the presence of the Sh^{DeliDura} allele and the absence of the sh^{MPOB}, or sh^{AVROS} alleles, and thus predict that the fruit form of the plant is dura. Alternatively, if there is no cleavage, then the Sh^{DeliDura} allele is not detected, and the fruit form of the plant is predicted to be pisifera. Similarly, if partial cleavage is detected, then the presence of both Sh^{DeliDura} and a sh^{MPOB}, or sh^{AVROS} allele is indicated, and the fruit form of the plant is predicted to be tenera. In some cases, cleavage is compared to a positive control (e.g., active endonuclease with recognized SHELL locus or a portion thereof, or cleaved SHELL locus or a portion thereof) and/or a negative control (e.g., no endonuclease, non recognized SHELL locus, or no template nucleic acid). In some cases, cleavage patterns are compared to one or more nucleic acid samples (e.g., one or more DNA samples) that contain nucleic acids that are of or about the size of expected cleavage patterns. For example, cleavage patterns may be compared to a ladder of DNA size standards.

Cleavage can be detected by assaying for a change in the relative sizes of oil palm nucleic acid or a portion thereof (e.g., the SHELL locus or a portion thereof). For example, oil palm nucleic acid or a portion thereof (e.g., the SHELL locus or a portion thereof) can be contacted with one or more endonucleases in a reaction mixture, optionally amplified, the reaction mixture loaded onto an agarose or acrylamide gel, electrophoresed, and the relative sizes of the nucleic acids visualized or otherwise detected. The electrophoresis can be slab gel electrophoresis or capillary electrophoresis. Cleavage can also be detected by assaying for successful amplification of the oil palm nucleic acid or a portion thereof (e.g., the SHELL locus or a portion thereof). For example, oil palm nucleic acid or a portion thereof (e.g., the SHELL locus or a portion thereof) can be contacted with one or more endonucleases in a reaction mixture, amplified, the reaction mixture loaded onto an agarose or acrylamide gel, electrophoresed, and the presence or absence of one or more amplicons, or the relative sizes of amplicons visualized or otherwise detected.

Detection of cleavage products can be quantitative or semi-quantitative. For example, visualization or other detection can include detection of fluorescent dyes intercalated into double stranded DNA. In such cases, the fluorescent signal is proportional to both the size of the fluorescent DNA molecule and the molar quantity. Thus, after correction for the size of the DNA molecule, the relative molar quantities of cleavage products can be compared. In some cases, quantitative detection provides discrimination between partial and complete cleavage or discrimination between a plant that is homozygous at the SHELL locus or heterozygous at the SHELL locus.

2. Oligonucleotide Detection

In other embodiments, contacting the oil palm nucleic acid with a detection reagent includes contacting the oil palm nucleic acid or a portion thereof (e.g., the SHELL locus or a portion thereof) with an oligonucleotide specific for one or more SHELL alleles (e.g., specific for an sh^{DeliDura}, sh^{MPOB}, or sh^{AVROS} allele) under conditions which allow for specific hybridization to the one or more SHELL alleles or specific cleavage of the one or more SHELL alleles. Such conditions can include stringent conditions as described herein. Such conditions can also include conditions that allow specific priming of polymerization by the hybridized oligonucleotide at the SHELL locus. Detection of hybridization, cleavage, or polymerization can then indicate the presence of the one or more SHELL alleles that the oligonucleotide is specific for. For example, if the oligonucleotide is specific for the Sh^{DeliDura} allele, then detection of hybridization can indicate the presence of the Sh^{DeliDura} allele and predict that the fruit form of the plant is dura or tenera. Alternatively, if the Sh^{DeliDura} allele is not detected, the fruit form of the plant is predicted to be pisifera. Hybridization can be detected by assaying for the presence of the oligonucleotide, the presence of a label linked to the oligonucleotide, or assaying for polymerization of the oligonucleotide. Polymerization of the oligonucleotide can be detected by assaying for amplification as described herein.

Polymerization of the oligonucleotide can also be detected by assaying for the incorporation of a detectable label during the polymerization process. For example, a primer extension assay can be performed. Primer extension is a two-step process that first involves the hybridization of a probe to the bases immediately upstream of a nucleotide polymorphism, such as the polymorphisms that give rise to the Sh^{DeliDura}, sh^{MPOB}, and sh^{AVROS} genotypes, followed by a ‘mini-sequencing’ reaction, in which DNA polymerase extends the hybridized primer by adding bases that are complementary to one or more of the polymorphic sequences. At each position, incorporated bases are detected and the identity of the allele is determined. Because primer extension is based on the highly accurate DNA polymerase enzyme, the method is generally very reliable. Primer extension is able to genotype most polymorphisms under very similar reaction conditions making it also highly flexible. The primer extension method is used in a number of assay formats. These formats use a wide range of detection techniques that include fluorescence, chemiluminescence, directly sensing the ions produced by template-directed DNA polymerase synthesis, MALDI-TOF Mass spectrometry and ELISA-like methods.

Primer extension reactions can be performed with either fluorescently labeled dideoxynucleotides (ddNTP) or fluorescently labeled deoxynucleotides (dNTP). With ddNTPs, probes hybridize to the target DNA immediately upstream of polymorphism, and a single, ddNTP complementary to at least one of alleles is added to the 3′ end of the probe (the missing 3′-hydroxyl in didioxynucleotide prevents further nucleotides from being added). Each ddNTP is labeled with a different fluorescent signal allowing for the detection of all four possible single nucleotide variations in the same reaction. The reaction can be performed in a multiplex reaction (for simultaneous detection of multiple polymorphisms) by using primers of different lengths and detecting fluorescent signal and length. With dNTPs, allele-specific probes have 3′ bases which are complementary to each of the possible nucleotides to be detected. If the target DNA contains a nucleotide complementary to the probe's 3′ base, the target DNA will completely hybridize to the probe, allowing DNA polymerase to extend from the 3′ end of the probe. This is detected by the incorporation of the fluorescently labeled dNTPs onto the end of the probe. If the target DNA does not contain a nucleotide complementary to the probe's 3′ base, the target DNA will produce a mismatch at the 3′ end of the probe and DNA polymerase will not be able to extend from the 3′ end of the probe. In this case, several labeled dNTPs may get incorporated into the growing strand, allowing for increased signal. Exemplary primer extension methods and compositions include the SNaPshot method. Primer extension reactions can also be performed using a mass spectrometer. The extension reaction can use ddNTPs as above, but the detection of the allele is dependent on the actual mass of the extension product and not on a fluorescent molecule.

In some cases, two oligonucleotides with differing specificities for one or more SHELL alleles are contacted with oil palm nucleic acid or a portion thereof (e.g., the SHELL locus or a portion thereof). In some cases, the two oligonucleotides are differentially labeled. In such cases, the contacting can be performed in a single reaction, and hybridization can be differentially detected. Alternatively, the two or more oligonucleotides can be contacted with oil palm nucleic acid that has been separated into two or more reactions, such that each reaction can be contacted with a different oligonucleotide. As yet another alternative, the two or more oligonucleotides can be hybridized to oil palm nucleic in a single reaction, polymerization or amplification performed at the SHELL locus, and the amplification or polymerization of the SHELL alleles can be differentially detected. For example, the two or more oligonucleotides can be blocking oligonucleotides such that amplification does not substantially occur when the oligonucleotide is bound. As another example, the two or more oligonucleotides can contain a fluorophore and a quencher, such that amplification of the specifically bound oligonucleotide degrades the oligonucleotide and provides an increase in fluorescent signal. As yet another example, polymerization or amplification can provide polymerization/amplification products of a size that is allele specific. In some cases, one or more control reactions are also included, such as a no-oligonucleotide control, or a positive control containing one or more of Sh^{DeliDura}, sh^{MPOB}, or sh^{AVROS} nucleic acid.

For example, an oligonucleotide specific for the Sh^{DeliDura} allele, and an oligonucleotide specific for the sh^{MPOB} or sh^{AVROS} allele can be contacted with oil palm nucleic acid under stringent conditions. Unbound oligonucleotide and/or nucleic acid can then be washed away. Hybridization can then be detected. Hybridization of only the first oligonucleotide would indicate the presence of the Sh^{DeliDura} allele, and thus predict a dura phenotype. Hybridization of only the second oligonucleotide would indicate the presence of the sh^{MPOB} or sh^{AVROS} allele, and thus predict a pisifera phenotype. Hybridization of both oligonucleotides would indicate the presence of both a Sh^{DeliDura} allele and either the sh^{MPOB} or sh^{AVROS} allele, and thus predict a tenera shell fruit form.

As another example, oil palm nucleic acid can be contacted with three oligonucleotides in three different reaction mixtures. The first oligonucleotide can be capable of specifically hybridizing to the Sh^{DeliDura} allele. The second oligonucleotide can be capable of specifically hybridizing to the sh^{MPOB} allele. The third oligonucleotide can be capable of specifically hybridizing to the sh^{AVROS} allele. The reaction mixtures can optionally contain another oligonucleotide that specifically hybridizes to the a sequence in the oil palm genome and in combination with any of the first second and third oligonucleotide primers flanks a region, e.g., about 10, 25, 50, 100, 150, 200, 250, 300, 350, 500, 600, 750, 1000, 2000, 5000, 7500, 10000 or more continuous nucleotides, of the oil palm genome at or near the SHELL locus. The first, second, and third oligonucleotides can then be polymerized and the presence or absence of polymerization product detected. For example, PCR can be performed. In some cases, the presence or absence of polymerization product is detected by detection of amplification. In some cases, the presence or absence of polymerization product is detected by detection of a label incorporated during the polymerization.

Detection of a polymerization product of the first oligonucleotide would indicate the presence of the Sh^{DeliDura} allele. Detection of a polymerization product of the second oligonucleotide would indicate the presence of the sh^{MPOB} allele. Detection of a polymerization product of the third oligonucleotide would indicate the presence of the sh^{AVROS} allele. Thus, the six prevalent SHELL genotypes can be detected and the three resulting phenotypes predicted. In some cases, the polymerization and/or detection can be quantitative or semi-quantitative such that homozygous and heterozygous plants can be distinguished. For example, oil palm nucleic acid can be contacted with the first oligonucleotide, polymerized, and the polymerization detected quantitatively. Absence of polymerization can indicate absence of the Sh^{DeliDura} allele and predict a pisifera phenotype. A quantitative polymerization signal that indicates both heterozygosity and the presence of the Sh^{DeliDura} allele can predict a tenera phenotype. And a signal that indicates the plant is homozygous Sh^{DeliDura} can predict a dura phenotype.

As the allele-specific differences in the SHELL gene are SNPs, methods useful for SNP detection can also be used to detect the SHELL alleles. The amount and/or presence of an allele of a SNP in a sample from an individual can be determined using many detection methods that are well known in the art. A number of SNP assay formats entail one of several general protocols: hybridization using allele-specific oligonucleotides, primer extension, allele-specific ligation, sequencing, or electrophoretic separation techniques, e.g., singled-stranded conformational polymorphism (SSCP) and heteroduplex analysis. Exemplary assays include 5′ nuclease assays, template-directed dye-terminator incorporation, molecular beacon allele-specific oligonucleotide assays, single-base extension assays, and SNP scoring by real-time pyrophosphate sequences. Analysis of amplified sequences can be performed using various technologies such as microchips, fluorescence polarization assays, and matrix-assisted laser desorption ionization (MALDI) mass spectrometry. Two methods that can also be used are assays based on invasive cleavage with Flap nucleases and methodologies employing padlock probes.

Determining the presence or absence of a particular SNP allele is generally performed by analyzing a nucleic acid sample that is obtained from a biological sample from the individual to be analyzed. While the amount and/or presence of a SNP allele can be directly measured using RNA from the sample, often times the RNA in a sample will be reverse transcribed, optionally amplified, and then the SNP allele will be detected in the resulting cDNA.

Frequently used methodologies for analysis of nucleic acid samples to measure the amount and/or presence of an allele of a SNP are briefly described. However, any method known in the art can be used in the invention to measure the amount and/or presence of single nucleotide polymorphisms.

3. Allele Specific Hybridization

This technique, also commonly referred to as allele specific oligonucleotide hybridization (ASO) (e.g., Stoneking et al., Am. J. Hum. Genet. 48:70-382, 1991; Saiki et al., Nature 324, 163-166, 1986; EP 235,726; and WO 89/11548), relies on distinguishing between two DNA molecules differing by one base by hybridizing an oligonucleotide probe that is specific for one of the variants to an amplified product obtained from amplifying the nucleic acid sample. In some embodiments, this method employs short oligonucleotides, e.g., 15-20 bases in length. The probes are designed to differentially hybridize to one variant versus another. Principles and guidance for designing such probe is available in the art, e.g., in the references cited herein. Hybridization conditions should be sufficiently stringent that there is a significant difference in hybridization intensity between alleles, and preferably an essentially binary response, whereby a probe hybridizes to only one of the alleles. Some probes are designed to hybridize to a segment of target DNA or cDNA such that the polymorphic site aligns with a central position (e.g., within 4 bases of the center of the oligonucleotide, for example, in a 15-base oligonucleotide at the 7 position; in a 16-based oligonucleotide at either the 8 or 9 position) of the probe (e.g., a polynucleotide of the invention distinguishes between two SNP alleles as set forth herein), but this design is not required.

The amount and/or presence of an allele is determined by measuring the amount of allele-specific oligonucleotide that is hybridized to the sample. Typically, the oligonucleotide is labeled with a label such as a fluorescent label. For example, an allele-specific oligonucleotide is applied to immobilized oligonucleotides representing potential SNP sequences. After stringent hybridization and washing conditions, fluorescence intensity is measured for each SNP oligonucleotide.

In one embodiment, the nucleotide present at the polymorphic site is identified by hybridization under sequence-specific hybridization conditions with an oligonucleotide probe exactly complementary to one of the polymorphic alleles in a region encompassing the polymorphic site. The probe hybridizing sequence and sequence-specific hybridization conditions are selected such that a single mismatch at the polymorphic site destabilizes the hybridization duplex sufficiently so that it is effectively not formed. Thus, under sequence-specific hybridization conditions, stable duplexes will form only between the probe and the exactly complementary allelic sequence. Thus, oligonucleotides from about 10 to about 35 nucleotides in length, e.g., from about 15 to about 35 nucleotides in length, which are exactly complementary to an allele sequence in a region which encompasses the polymorphic site (e.g., SEQ ID NO:1, 2, 3, or 4) are within the scope of the invention.

In an alternative embodiment, the amount and/or presence of the nucleotide at the polymorphic site is identified by hybridization under sufficiently stringent hybridization conditions with an oligonucleotide substantially complementary to one of the SNP alleles in a region encompassing the polymorphic site, and exactly complementary to the allele at the polymorphic site. Because mismatches that occur at non-polymorphic sites are mismatches with both allele sequences, the difference in the number of mismatches in a duplex formed with the target allele sequence and in a duplex formed with the corresponding non-target allele sequence is the same as when an oligonucleotide exactly complementary to the target allele sequence is used. In this embodiment, the hybridization conditions are relaxed sufficiently to allow the formation of stable duplexes with the target sequence, while maintaining sufficient stringency to preclude the formation of stable duplexes with non-target sequences. Under such sufficiently stringent hybridization conditions, stable duplexes will form only between the probe and the target allele. Thus, oligonucleotides from about 10 to about 35 nucleotides in length, preferably from about 15 to about 35 nucleotides in length, which are substantially complementary to an allele sequence in a region which encompasses the polymorphic site, and are exactly complementary to the allele sequence at the polymorphic site, are within the scope of the invention.

The use of substantially, rather than exactly, complementary oligonucleotides may be desirable in assay formats in which optimization of hybridization conditions is limited. For example, in a typical multi-target immobilized-probe assay format, probes for each target are immobilized on a single solid support. Hybridizations are carried out simultaneously by contacting the solid support with a solution containing target DNA or cDNA. As all hybridizations are carried out under identical conditions, the hybridization conditions cannot be separately optimized for each probe. The incorporation of mismatches into a probe can be used to adjust duplex stability when the assay format precludes adjusting the hybridization conditions. The effect of a particular introduced mismatch on duplex stability is well known, and the duplex stability can be routinely both estimated and empirically determined, as described above. Suitable hybridization conditions, which depend on the exact size and sequence of the probe, can be selected empirically using the guidance provided herein and well known in the art. The use of oligonucleotide probes to detect single base pair differences in sequence is described in, for example, Conner et al., 1983, Proc. Natl. Acad. Sci. USA 80:278-282, and U.S. Pat. Nos. 5,468,613 and 5,604,099, each incorporated herein by reference.

The proportional change in stability between a perfectly matched and a single-base mismatched hybridization duplex depends on the length of the hybridized oligonucleotides. Duplexes formed with shorter probe sequences are destabilized proportionally more by the presence of a mismatch. In practice, oligonucleotides between about 15 and about 35 nucleotides in length are preferred for sequence-specific detection. Furthermore, because the ends of a hybridized oligonucleotide undergo continuous random dissociation and re-annealing due to thermal energy, a mismatch at either end destabilizes the hybridization duplex less than a mismatch occurring internally. Preferably, for discrimination of a single base pair change in target sequence, the probe sequence is selected which hybridizes to the target sequence such that the polymorphic site occurs in the interior region of the probe.

The above criteria for selecting a probe sequence that hybridizes to a particular SNP apply to the hybridizing region of the probe, i.e., that part of the probe which is involved in hybridization with the target sequence. A probe may be bound to an additional nucleic acid sequence, such as a poly-T tail used to immobilize the probe, without significantly altering the hybridization characteristics of the probe. One of skill in the art will recognize that for use in the present methods, a probe bound to an additional nucleic acid sequence which is not complementary to the target sequence and, thus, is not involved in the hybridization, is essentially equivalent to the unbound probe.

Suitable assay formats for detecting hybrids formed between probes and target nucleic acid sequences in a sample are known in the art and include the immobilized target (dot-blot) format and immobilized probe (reverse dot-blot or line-blot) assay formats. Dot blot and reverse dot blot assay formats are described in U.S. Pat. Nos. 5,310,893; 5,451,512; 5,468,613; and 5,604,099; each incorporated herein by reference.

In a dot-blot format, amplified target DNA or cDNA is immobilized on a solid support, such as a nylon membrane. The membrane-target complex is incubated with labeled probe under suitable hybridization conditions, unhybridized probe is removed by washing under suitably stringent conditions, and the membrane is monitored for the presence of bound probe.

In the reverse dot-blot (or line-blot) format, the probes are immobilized on a solid support, such as a nylon membrane or a microtiter plate. The target DNA or cDNA is labeled, typically during amplification by the incorporation of labeled primers. One or both of the primers can be labeled. The membrane-probe complex is incubated with the labeled amplified target DNA or cDNA under suitable hybridization conditions, unhybridized target DNA or cDNA is removed by washing under suitably stringent conditions, and the membrane is monitored for the presence of bound target DNA or cDNA.

An allele-specific probe that is specific for one of the polymorphism variants is often used in conjunction with the allele-specific probe for the other polymorphism variant. In some embodiments, the probes are immobilized on a solid support and the target sequence in an individual is analyzed using both probes simultaneously. Examples of nucleic acid arrays are described by WO 95/11995. The same array or a different array can be used for analysis of characterized polymorphisms. WO 95/11995 also describes sub-arrays that are optimized for detection of variant forms of a pre-characterized polymorphism.

In some embodiments, allele-specific oligonucleotide probes can be utilized in a branched DNA assay to differentially detect SHELL alleles. For example, allele-specific oligonucleotide probes can be used as capture extender probes that hybridize to a capture probe and SHELL in an allele specific manner. Label extenders can then be utilized to hybridize to SHELL in a non allele-specific manner and to an amplifier (e.g., alkaline phosphatase). In some cases, a pre-amplifier molecule can further increase signal by binding to the label extender and a plurality of amplifiers. As another example, non allele-specific capture extender probes can be used to capture SHELL, and allele-specific label extenders can be used to differentially detect SHELL alleles. In some cases, the capture extender probes and/or label extenders hybridize to allele specific SHELL cleavage sites (e.g., hybridize to an Eco57I or HindIII site). In some cases, the probes do not hybridize to SHELL DNA that has been cleaved with an allele specific endonuclease (e.g., Eco57I or HindIII, or an isoschizomer thereof).

4. Allele-Specific Primers

The amount and/or presence of an allele is also commonly detected using allele-specific amplification or primer extension methods. These reactions typically involve use of primers that are designed to specifically target a polymorphism via a mismatch at the 3′ end of a primer. The presence of a mismatch affects the ability of a polymerase to extend a primer when the polymerase lacks error-correcting activity. For example, to detect an allele sequence using an allele-specific amplification- or extension-based method, a primer complementary to the polymorphic nucleotide of a SNP is designed such that the 3′ terminal nucleotide hybridizes at the polymorphic position. The presence of the particular allele can be determined by the ability of the primer to initiate extension. If the 3′ terminus is mismatched, the extension is impeded. If a primer matches the polymorphic nucleotide at the 3′ end, the primer will be efficiently extended.

The primer can be used in conjunction with a second primer in an amplification reaction. The second primer hybridizes at a site unrelated to the polymorphic position. Amplification proceeds from the two primers leading to a detectable product signifying the particular allelic form is present. Allele-specific amplification- or extension-based methods are described in, for example, WO 93/22456; U.S. Pat. Nos. 5,137,806; 5,595,890; 5,639,611; and U.S. Pat. No. 4,851,331.

Using allele-specific amplification-based methods, identification and/or quantification of the alleles require detection of the presence or absence of amplified target sequences. Methods for the detection of amplified target sequences are well known in the art. For example, gel electrophoresis and probe hybridization assays described are often used to detect the presence of nucleic acids.

In an alternative probe-less method, the amplified nucleic acid is detected by monitoring the increase in the total amount of double-stranded DNA in the reaction mixture, is described, e.g., in U.S. Pat. No. 5,994,056; and European Patent Publication Nos. 487,218 and 512,334. The detection of double-stranded target DNA or cDNA relies on the increased fluorescence various DNA-binding dyes, e.g., SYBR Green, exhibit when bound to double-stranded DNA.

Allele-specific amplification methods can be performed in reactions that employ multiple allele-specific primers to target particular alleles. Primers for such multiplex applications are generally labeled with distinguishable labels or are selected such that the amplification products produced from the alleles are distinguishable by size. Thus, for example, both alleles in a single sample can be identified and/or quantified using a single amplification by various methods.

As in the case of allele-specific probes, an allele-specific oligonucleotide primer may be exactly complementary to one of the polymorphic alleles in the hybridizing region or may have some mismatches at positions other than the 3′ terminus of the oligonucleotide, which mismatches occur at non-polymorphic sites in both allele sequences.

5. Amplification

Amplification includes any method in which nucleic acid is reproduced, copied, or amplified. In some cases, the amplification produces a copy of the template nucleic acid. In other cases, the amplification produces a copy of a portion of the template nucleic acid (e.g., a copy of the SHELL locus or a portion thereof). Amplification methods include the polymerase chain reaction (PCR), the ligase chain reaction (LCR), self-sustained sequence replication (3SR), the transcription based amplification system (TAS), nucleic acid sequence-based amplification (NASBA), strand displacement amplification (SDA), rolling circle amplification (RCA), hyper-branched RCA (HRCA), helicase-dependent DNA amplification (HDA), single primer isothermal amplification, signal-mediated amplification of RNA technology (SMART), loop-mediated isothermal amplification (LAMP), isothermal multiple displacement amplification (IMDA), and circular helicase-dependent amplification (cHDA). The amplification reaction can be isothermal, or can require thermal cycling. Isothermal amplification methods, include but are not limited to, TAS, NASBA, 3SR, SMART, SDA, RCA, LAMP, IMDA, HDA, SPIA, and cHDA. Methods and compositions for isothermal amplification are provided in, e.g., Gill and Ghaemi, Nucleosides, Nucleotides, and Nucleic Acids, 27: 224-43 (2008).

Loop-mediated isothermal amplification (LAMP) is described in, e.g., Notomi, et al., Nucleic Acids Research, 28(12), e63 i-vii, (2000). The method produces large amounts of amplified DNA in a short period of time. In some cases, successful LAMP amplification can produce pyrophosphate ions in sufficient amount to alter the turbidity, or color of the reaction solution. Thus, amplification can be assayed by observing an increase in turbidity, or a change in the color of the sample. Alternatively, amplified DNA can be observed using any amplification detection method including detecting intercalation of a fluorescent dye and/or gel or capillary electrophoresis.

In some cases, the loop-mediated isothermal amplification (LAMP) is performed with four primers or three or more sets of four primers for amplification of the SHELL gene, or a portion thereof, including a forward inner primer, a forward outer primer, a backward inner primer, and a backward outer primer. In some cases, one, two, or more additional primers can be used to identify multiple regions or alleles in the same reaction. In some cases, LAMP can be performed with a set of Sh^{DeliDura} specific primers, a set of sh^{MPOB} specific primers, and/or a set of sh^{AVROS} specific primers. In some cases, LAMP can be performed with a set of primers that amplifies the Sh^{DeliDura}, sh^{MPOB} and sh^{AVROS} alleles or a portion thereof.

For example, oil palm plant DNA can be analyzed by LAMP in three or four separate reaction mixtures. In one reaction mixture, oil palm plant DNA is amplified using Sh^{DeliDura} specific LAMP primers. In another reaction mixture, oil palm plant DNA is amplified using sh^{MPOB} specific LAMP primers. In a third reaction mixture, oil palm plant DNA is amplified using sh^{AVROS} specific LAMP primers. In some cases, the oil palm plant DNA is contacted with an allele specific endonuclease (e.g., Eco57I, HindIII, or an isoschizomer thereof) in one or more reaction mixtures. In some cases, a fourth reaction mixture can contain wild-type DNA and/or non allele specific primers as a positive control. In some cases, amplification indicates the presence of a specific SHELL allele or alleles in each reaction mixture. For example, an increase in turbidity of the sample, an increase in fluorescence of an intercalating dye, or a change in color of the sample can indicate amplification in a reaction mixture and thus the presence of a specific SHELL allele or alleles. In still other cases, lack of amplification indicates the presence of a specific SHELL allele or alleles in each reaction mixture. In some cases, the amplification products are visualized (e.g., gel or capillary electrophoresis). Cleavage patterns indicative of SHELL genotype are thus determined.

As another example, oil palm plant DNA can be analyzed in two, three, or four separate reaction mixtures by contacting one reaction mixture with an allele specific endonuclease (e.g., Eco57I or an isoschizomer thereof), and another reaction mixture with a different allele specific endonuclease (e.g., HindIII or an isoschizomer thereof). Optionally, a third reaction mixture can contain a no enzyme control. Optionally, a fourth reaction mixture can contain an oil palm plant DNA control (e.g., can contain wild-type oil palm plant DNA or a portion thereof, or tenera, or pisifera DNA). LAMP primers can be used to amplify the SHELL locus or a portion thereof. In some cases, amplification indicates the presence of a specific SHELL allele or alleles in each reaction mixture. For example, an increase in turbidity or fluorescence of an intercalating dye, or a change in color can indicate amplification in a reaction mixture and thus the presence of a specific SHELL allele or alleles. In still other cases, lack of amplification indicates the presence of a specific SHELL allele or alleles in each reaction mixture. In some cases, the amplification products are visualized (e.g., gel or capillary electrophoresis). Cleavage patterns indicative of SHELL genotype are thus determined.

In some cases, one or more LAMP primers hybridizes to an allele specific cleavage site, e.g., an Eco57I or HindIII cleavage site.

Amplification, e.g., any of the amplification methods described herein, can be performed using a hybridized oligonucleotide detection reagent as a primer, such that one or more SHELL alleles are specifically amplified. Alternatively, amplification can be performed using a primer or set of primers that does not distinguish between SHELL alleles. As yet another alternative, amplification can be performed such that the different SHELL alleles provide amplicons that can be differentially detected. For example, the amplicons can differ in size among the SHELL alleles or be differentially labeled (e.g. be attached to a different fluorophore). As yet another alternative, amplification can be performed such that cleaved SHELL alleles are not amplified, but uncleaved SHELL alleles are amplified.

In some cases, SHELL alleles can be detected by portioning oil palm plant DNA into three reactions, and optionally one or more control reactions. For example, one reaction can contain a Sh^{DeliDura} allele-specific amplification primer, primers, or primer sets. A second reaction can contain a sh^{Avros} allele-specific amplification primer, primers, or primer sets. A third reaction can contain a sh^{MPOB} allele-specific amplification primer, primers, or primer sets. Successful amplification in the first reaction indicates the presence of an Sh^{DeliDura} allele. Successful amplification in the second reaction indicates the presence of an sh^{Avros} allele. Successful amplification in the third reaction indicates the presence of an sh^{MPOB} allele. Thus, all six genotypes can be detected and all three possible fruit form phenotypes predicted.

Amplification detection can include end-point detection and real-time detection. End-point detection can include agarose or acrylamide gel electrophoresis and visualization. For example, amplification can be performed on template nucleic acid that has been contacted with one or more detection reagents (e.g., one or more endonucleases), and then the reaction mixture (or a portion thereof) can be loaded onto an acrylamide or agarose gel, electrophoresed, and the relative sizes of amplicons or the presence or absence of amplicons detected. Alternatively, amplification can be performed, amplicons contacted with one or more detection reagents (e.g., one or more endonucleases), and then the reaction mixture (or a portion thereof) can be loaded onto an acrylamide or agarose gel, electrophoresed, and the relative sizes of amplicons or the presence or absence of amplicons detected. Electrophoresis can include slab gel electrophoresis and capillary electrophoresis.

Real-time detection of amplification can include detection of the incorporation of intercalating dyes into accumulating amplicons, detection of fluorogenic nuclease activity, and detection of structured probes. The use of intercalating dyes utilizes fluorogenic compounds that only bind to double stranded DNA. In this type of approach, amplification product (which in some cases is double stranded) binds dye molecules in solution to form a complex. With the appropriate dyes, it is possible to distinguish between dye molecules remaining free in solution and dye molecules bound to amplification product. For example, certain dyes fluoresce efficiently only when bound to double stranded DNA, such as amplification product. Examples of such dyes include, but are not limited to, SYBR Green and Pico Green (from Molecular Probes, Inc., Eugene, Oreg.), ethidium bromide, propidium iodide, chromomycin, acridine orange, Hoechst 33258, TOTO-I, YOYO-1, and DAPI (4′,6-diamidino-2-phenylindole hydrochloride). Additional discussion regarding the use of intercalation dyes is provided, e.g., by Zhu et al., Anal. Chem. 66:1941-1948 (1994).

Fluorogenic nuclease assays are another example of a product quantification method that can be used successfully with the devices and methods described herein. The basis for this method of monitoring the formation of amplification product is to measure PCR product accumulation using a dual-labeled fluorogenic oligonucleotide probe, an approach frequently referred to in the literature as the “TaqMan” method.

The probe used in such assays can be a short (e.g. approximately 20-25 bases in length) polynucleotide that is labeled with two different fluorescent dyes. In some cases, the 5′ terminus of the probe can be attached to a reporter dye and the 3′ terminus attached to a quenching moiety. In other cases, the dyes can be attached at other locations on the probe. The probe can be designed to have at least substantial sequence complementarity with the probe-binding site on the target nucleic acid. Upstream and downstream PCR primers that bind to regions that flank the probe binding site can also be included in the reaction mixture. When the fluorogenic probe is intact, energy transfer between the fluorophore and quencher moiety occurs and quenches emission from the fluorophore. During the extension phase of PCR, the probe is cleaved, e.g., by the 5′ nuclease activity of a nucleic acid polymerase such as Taq polymerase, or by a separately provided nuclease activity that cleaves bound probe, thereby separating the fluorophore and quencher moieties. This results in an increase of reporter emission intensity that can be measured by an appropriate detector. Additional details regarding fluorogenic methods for detecting PCR products are described, for example, in U.S. Pat. No. 5,210,015 to Gelfand, U.S. Pat. No. 5,538,848 to Livak, et al, and U.S. Pat. No. 5,863,736 to Haaland, each of which is incorporated by reference in its entirety, as well as Heid, C. A., et al., Genome Research, 6:986-994 (1996); Gibson, U. E. M, et al., Genome Research 6:995-1001 (1996); Holland, P. M., et al, Proc. Natl. Acad. Sci. USA 4 88:7276-7280, (1991); and Livak, K. J., et al., PCR Methods and Applications 357-362 (1995).

Structured probes (e.g., “molecular beacons”) provide another method of detecting accumulated amplification product. With molecular beacons, a change in conformation of the probe as it hybridizes to a complementary region of the amplified product results in the formation of a detectable signal. In addition to the target-specific portion, the probe includes additional sections, generally one section at the 5′ end and another section at the 3′ end, that are complementary to each other. One end section is typically attached to a reporter dye and the other end section is usually attached to a quencher dye. In solution, the two end sections can hybridize with each other to form a stem loop structure. In this conformation, the reporter dye and quencher are in sufficiently close proximity that fluorescence from the reporter dye is effectively quenched by the quencher. Hybridized probe, in contrast, results in a linearized conformation in which the extent of quenching is decreased. Thus, by monitoring emission changes for the reporter dye, it is possible to indirectly monitor the formation of amplification product. Probes of this type and methods of their use is described further, for example, by Piatek, A. S., et al., Nat. Biotechnol. 16:359-63 (1998); Tyagi, S. and Kramer, F. R., Nature Biotechnology 14:303-308 (1996); and Tyagi, S. et al., Nat. Biotechnol. 16:49-53 (1998).

Detection of amplicons can be quantitative or semi-quantitative whether performed as a real-time analysis or as an end-point analysis. In general, the detection signal (e.g., fluorescence) is proportional to the molar quantity of the amplicon. Thus, the relative molar quantities of amplicons can be compared. In some cases, quantitative detection provides discrimination between a plant that is homozygous at the SHELL locus or heterozygous at the SHELL locus.

As described herein, hybridization, cleavage, and amplification methods can be combined. For example, oil palm plant nucleic acid can be hybridized to one or more oligonucleotides, cleaved and then amplified. Alternatively, oil palm plant nucleic acid can be amplified, cleaved, and then amplified again, or the cleavage products detected by hybridization with an oligonucleotide detection reagent.

### B. Sorting

In some embodiments, a seed or plant shell fruit form is predicted, and the seed or plant is sorted based on the predicted phenotype. For example, the seed or plant can be sorted into tenera, pisifera, and dura seeds or plants based on their predicted phenotype. Pisifera and dura seeds or plants can be sorted and stored separately as breeding stock for the generation of tenera plants. Tenera seeds or plants can be planted and cultivated for the enhanced oil yield they provide. In some cases, the plant is a seed and the sorting is performed on the seed. Alternatively, the plant is a seedling and the sorting is performed on the seedling before it is planted in the field or before its use in breeding. As yet another alternative, oil palm plants that have been planted in the field for optimal palm oil yield, but are not mature enough to verify shell fruit form can be assayed and pisifera and dura plants can be removed from the field. As yet another alternative, oil palm plants that have been planted in the field to maintain pisifera lines for breeding programs, but are not mature enough to verify shell fruit form can be assayed and dura plants can be removed from the field (tenera and pisifera palms carry one and two pisifera alleles respectively, whereas dura palms contain no pisifera alleles and do not contribute to the goal of pisifera allele maintenance). As yet another alternative, the shell fruit form is predicted from mature oil palm plants that have been planted in the field for cultivation, and are yielding fruit, yet and a more precise and simpler method of genetically determining the fruit form phenotype is preferred over traditional shell thickness measurements. Once the fruit form is determined, a palm is selected for a participation in a breeding program, or is selected for removal from the field based on the predicted fruit form phenotype.

### III. Kits

Described herein are kits for the prediction of shell fruit form of an oil palm plant. The kit can contain one or more endonucleases. In some cases, each endonuclease is specific for one or more SHELL alleles. For example, each endonuclease can recognize and cleave a sequence at or near one or more SHELL alleles, but does not recognize or cleave a sequence at or near at least one SHELL allele. In some cases, the one or more endonuclease is Eco57I, AcuI, or an isoschizomer thereof, HindIII, or an isoschizomer thereof. In some cases the kits comprise at least two endonucleases wherein the first endonuclease is Eco57I, AcuI, or an isoschizomer thereof, and the second endonuclease is HindIII or an isoschizomer thereof.

The kit can contain one or more oligonucleotide primers for amplification at or near the SHELL locus. For instance, the kit can include at least one primer that primes amplification of a portion of the SHELL gene comprising SEQ ID NO:1, 2, 3, or 4, or a primer pair that generates an amplicon comprising SEQ ID NO:1, 2, 3 or 4.

In other cases, the primer is specific for one or more SHELL alleles. For example, the primer can hybridize to, and prime polymerization of, a region at or near one or more SHELL alleles but does not hybridize to, or primer polymerization of, a region at or near one or more other SHELL alleles. In other cases, the primer can hybridize to, or prime polymerization of, a region at or near a HindIII or Eco57I site of a SHELL allele. In some cases, the oligonucleotide primer contains a nucleic acid of SEQ ID NOs:1-3 or a reverse complement thereof. In some cases, the primer can provide for amplification such as isothermal amplification or PCR.

In some cases, the kit can include a primer pair for amplification by, e.g. PCR or an isothermal amplification method. In some cases, the primer pair can specifically hybridize to the oil palm genome and flank at least about 8, 10, 12, 15, 20, 25, 30, 40, 50, 60, 70, 80, 90, 100, 125, 150, 175, 200, 250, 300, 350, 400, 450, 500, 600, 700, 800, 1000, 1500, 2000, 2500, 3000, 5000, 7500, or 10000 or more continuous nucleotides at or near the SHELL locus. The primer pair can specifically amplify one or more SHELL alleles and not amplify one or more SHELL alleles, or the primer pair can amplify all three naturally occurring SHELL alleles. In some cases, the primer pair contains SEQ ID NO:9 5′ TCAGCAGACAGAGGTGAAAG 3′, SEQ ID NO:10 5′ CCATTTGGATCAGGGATAAA 3′ or a reverse complement thereof.

The kit can also include control polynucleotides as described herein. For example, the kit can include one or more polynucleotides containing Sh^{DeliDura}, sh^{MPOB}, or sh^{AVROS} nucleic acid or a portion thereof (e.g., one or more nucleic acids that contain SEQ ID NOs: 1, 2, or 3). The kit can also include any of the reagents, proteins, oligonucleotides, etc. described herein. For instance, the control polynucleotides can be identical to expected amplicons based on the amplification primers described above (e.g., spanning the target sequence including SEQ ID NO:1, 2, 3, or 4), and/or portions of such amplicons that would occur upon cleavage with the endonucleases as described above. Thus, in some cases, the control polynucleotides include amplicons of Sh^{DeliDura}, sh^{MPOB}, or sh^{AVROS}, alleles either in separate containers or as a mixture, optionally in separate pre-cut (by the endonucleases above) versions. In some cases, control polynucleotides are a different nucleic acid sequence from the Sh^{DeliDura}, sh^{MPOB}, or sh^{AVROS} alleles or their expected amplicons, but of approximately the expected size.

### IV. Systems and Machines

Machines can be utilized to carry out one or more methods described herein, prepare plant samples for one or more methods described herein, or facilitate high throughput sorting of oil palm plants.

In some cases, a machine can sort and orient seeds such that the seed are all oriented in a similar manner. The seeds for example, can be oriented such that embryo region of the seed is down and the embryo free region is oriented up. In some cases, the seeds can be placed into an ordered array or into a single line.

A sample of endosperm material or fluid containing nucleic acid can be extracted from one or a plurality of oil palm seeds in a manner that does not damage the embryo. For example, endosperm material can be extracted from the sampling zone (see FIG. 4-A) with a needle or probe that penetrates the seed shell and enters the sampling zone and avoids the embryo containing zone (FIG. 4-B). The sampled material or fluid can further be purified from contaminating maternal DNA by removing fragments of the seed shell that might be present in the endosperm sample. In some cases, endosperm DNA can then be extracted from the endosperm material or fluid. Alternatively, the machine can obtain nucleic acid from a seedling, an immature (e.g., non fruit bearing) plant, or a mature plant.

Samples can be extracted by grinding, cutting, slicing, piercing, needle coring, needle aspiration or the like. In some embodiments, the sampling is controlled to remove a useful amount of tissue (e.g., endosperm) for analytical purposes without significant effect on viability potential of the sampled seed. For example, in some cases, sample extraction can reduce the number of viable (e.g., able to give rise to a plant) seeds in a population by less than about 20%, 15%, 10%, 5%, 2.5%, 1%, or less.

In some embodiments, the sampling is controlled to deter contamination of the sample. For example, washing steps can be employed between sample processing steps. Alternatively, disposable or removable sample handling elements can be utilized, e.g., disposable pipetting tips, disposable receptacles or containers, or disposable blades or grinders.

In some embodiments, the seed is held in pre-determined orientation to facilitate efficient and accurate sampling. For example, the machine can orient the seeds by seed shape or visual appearance. In some cases, the seed is oriented to facilitate sampling from the ‘Crown’ of each respective seed, containing the cotyledon and/or endosperm tissue of the seed, so that the germination viability of each seed is preserved.

In some cases, the machine can separately store plants or seeds and their extracted samples without reducing, or without substantially reducing the viability of the seeds. In some cases, the extracted samples and stored plants or seeds are organized, labeled, or catalogued in such a way that the sample and the seed from which it is derived can be determined. In some cases, the extracted samples and stored plants or seeds are tracked so that each can be accessed after data is collected. For example, a sample can be extracted from a seed and the SHELL genotype determined for the sample, and thus the seed. The seed can then be accessed and planted, stored, or destroyed based on the predicted fruit form phenotype.

In some cases, the extraction and storing are performed automatically by the machine, but the genotype analysis and/or treatment of analyzed seeds performed manually or performed by another machine. As such, in some embodiments, a system is provided consisting of two or more machines for extraction of seed samples, seed sorting and storing, and prediction of fruit form phenotype.

In some cases, the plants or seed are stored in an array by the machine, such as individually in an array of tubes or wells. The plants can be sampled and/or interrogated in or from each well. The results of the sampling or interrogating can be correlated with the position of the plant in the array.

Sampling can include extraction and/or analysis of nucleic acid (e.g., DNA or RNA), magnetic resonance imaging, optical dispersion, optical absorption, ELISA, enzymatic assay, or the like.

Systems, machines, methods and compositions for seed sampling and/or sorting are further described in, e.g., U.S. Pat. Nos. 6,307,123; 6,646,264; 7,367,155; 8,312,672; 7,685,768; 7,673,572; 8,443,545; 7,998,669; 8,362,317; 8,076,076; 7,402,731; 7,600,642; 8,237,016; 8,401,271; 8,281,935; 8,241,914; 6,880,771; 7,909,276; 8,221,968; and 7,454,989. Systems, machines, methods and compositions for seed sampling and/or sorting are also further described in, e.g., U.S. Patent Application Publication NOs: 2012/180386; 2009/070891; 2013/104454, 2012/117865, 2008/289061; 2008/000815; 2011/132721; 2011/195866; 2011/0079544; 2010/0143906; and 2013/079917. Additional systems, machines, methods, and compositions for seed sampling are further described in international patent application publications WO2011/119390; and WO2011/119394.

Also provided herein are methods for using the systems, machines, methods, and compositions described herein for seed sampling or sorting. For example, a seed or set of seeds can be loaded into a seed sampler, and a sample obtained. In some cases, the seed can be stored, e.g., in an array. In some cases, the storage is performed by the machine that samples the seed. In other cases, the seed is stored by another machine, or stored manually. In some cases, DNA can be extracted from the sample. In some cases, sample can be obtained and DNA extracted by the same machine. In other cases, the DNA is extracted by another machine, or manually. The extracted DNA can be analyzed and the SHELL genotype determined. In some cases, the extracted DNA is analyzed by the same machine, by another machine, or manually. In some cases, fruit form phenotype is predicted from the SHELL genotype by the machine, a different machine, or manually. In some cases, stored seeds can be disposed of (e.g., cultivated or destroyed) based on the SHELL genotype or predicted fruit form phenotype. In some cases, the seed is disposed of by the machine, a different machine, or manually.

In some cases, the seed or seeds are shipped from a customer to a service provider, analyzed, and returned. In some cases, only seeds with a predicted phenotype or phenotypes are returned. For example, only tenera, only pisifera, only dura, or a combination thereof are returned. In other cases, seeds are sampled, and the samples are shipped from a customer to a service provider for analysis. The customer can then utilize information provided by the analysis to dispose of the seeds.

In some cases, reagents, such as the compositions described herein are provided for sampling of seeds manually or automatically. For example, oligonucleotide primers or probes as described herein can be provided. As another example, endonucleases and primers can be provided herein. As another example, reaction mixtures containing reagents necessary for analysis of nucleic acid from an oil palm plant can be provided.

All patents, patent applications, and other publications, including GenBank Accession Numbers, cited in this application are incorporated by reference in the entirety for all purposes.

### EXAMPLES

The following examples are offered to illustrate, but not to limit the claimed invention.

### Example 1

### Assay for Determining SHELL Genotype and Predicting Shell Fruit Form

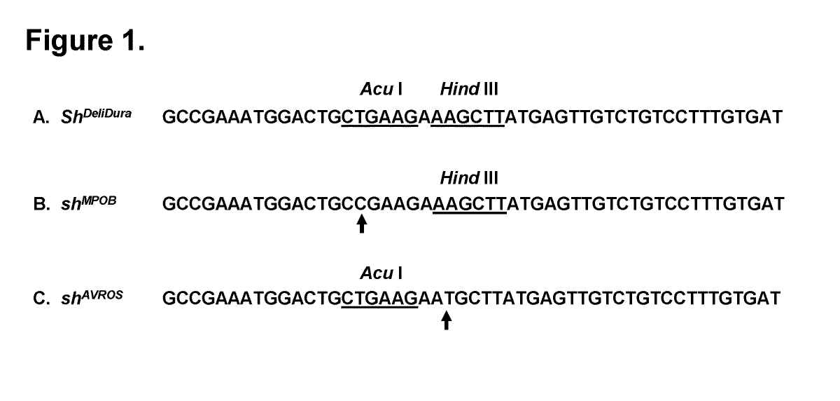

An approximately 350 bp amplicon, including SHELL exon 1, was amplified from genomic DNA extracted from oil palm leaf. A subset of this sequence, including the variant nucleotides, is shown in FIG. 1. Dura trees, seedlings or seeds are homozygous for the Sh^{DeliDura} allele, and the variant nucleotide positions (marked by arrows in FIG. 1-B and 1-C) retain an Eco57I/AcuI restriction enzyme recognition sequence (CTGAAG), including the leucine-coding codon that is mutated in the sh^{MPOB} allele, and a HindIII restriction enzyme recognition sequence (AAGCTT), including the lysine-coding codon that is mutated in the sh^{AVROS} allele. Pisifera trees, seedlings or seeds typically have one of three naturally occurring genotypes: i) homozygous for the sh^{MPOB} allele (lacking the Eco57I/AcuI recognition sequence), ii) homozygous for the sh^{AVROS} allele (lacking the HindIII recognition sequence) or iii) heterozygous sh^{MPOB}/sh^{AVROS}. Tenera trees, seedlings or seeds typically have one of two naturally occurring genotypes: i) heterozygous sh^{DeliDura}/sh^{MPOB} or ii) heterozygous sh^{DeliDura}/sh^{AVROS}.

SHELL exon 1 was PCR amplified under the following conditions: Genomic DNA from six oil palm trees of known genotype (approximately 10 ng each) was amplified in 1× FailSafe™ PCR Premix G (Epicentre), 6 μM forward primer, 6 μM reverse primer and 0.1 units Taq polymerase (Invitrogen) in a total volume of 20 μL. PCR primer sequences were SEQ ID NO:9 5′ TCAGCAGACAGAGGTGAAAG 3′ (forward) and SEQ ID NO:10 5′ CCATTTGGATCAGGGATAAA 3′ (reverse). PCR cycling conditions were 95° C. for 2 minutes, followed by 35 cycles of 94° C. for 30 seconds, 58.5° C. for 35 seconds and 72° C. for 2 minutes. A final incubation at 72° C. for 10 minutes was performed.

The PCR amplicon was split into three portions of equal DNA quantity. One portion was mock-treated (e.g., no endonuclease was added). The second portion was digested with AcuI, where 7.0 μL of PCR product was digested with 10 units of AcuI (New England Biolabs) in 1× CutSmart (New England Biolabs) for 1 hour at 37° C. in a total volume of 20 μL. The third portion was digested with HindIII, where 7.0 μL PCR product was digested with 10 units of HindIII (New England Biolabs) in 1×NEB Buffer 2 (New England Biolabs) for 1 hour at 37° C. in a total volume of 20 μL. Restriction digestion reactions were inactivated by incubating at 80° C. for 15 minutes.

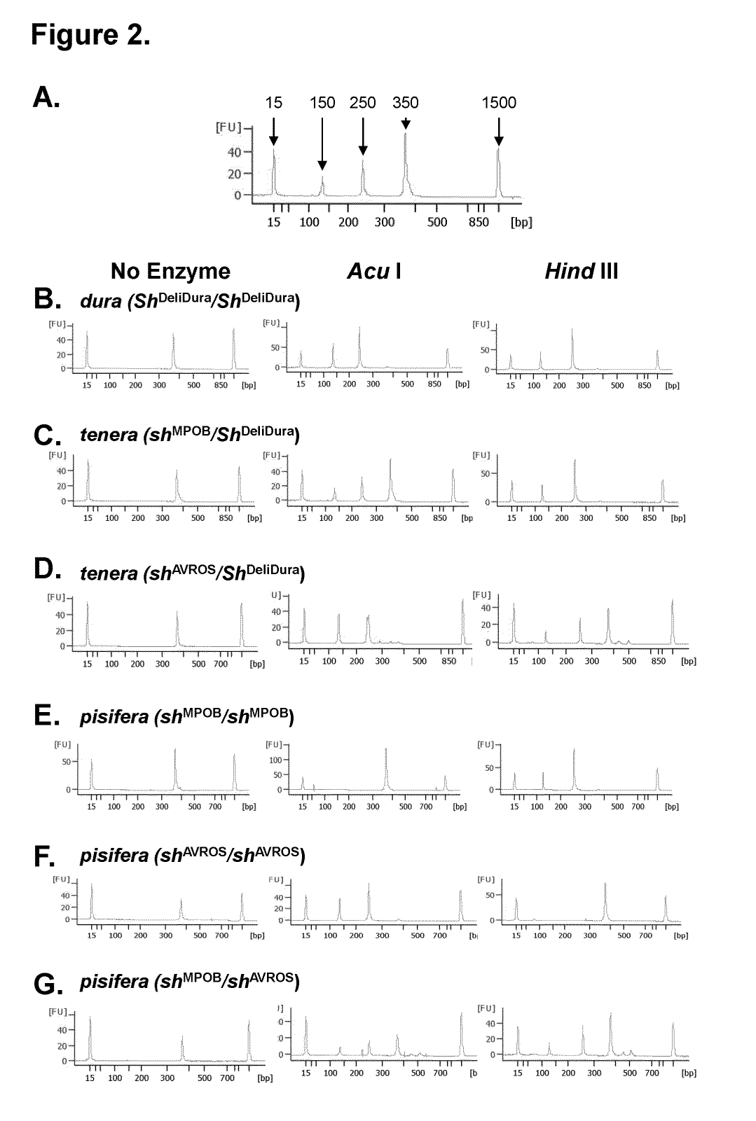

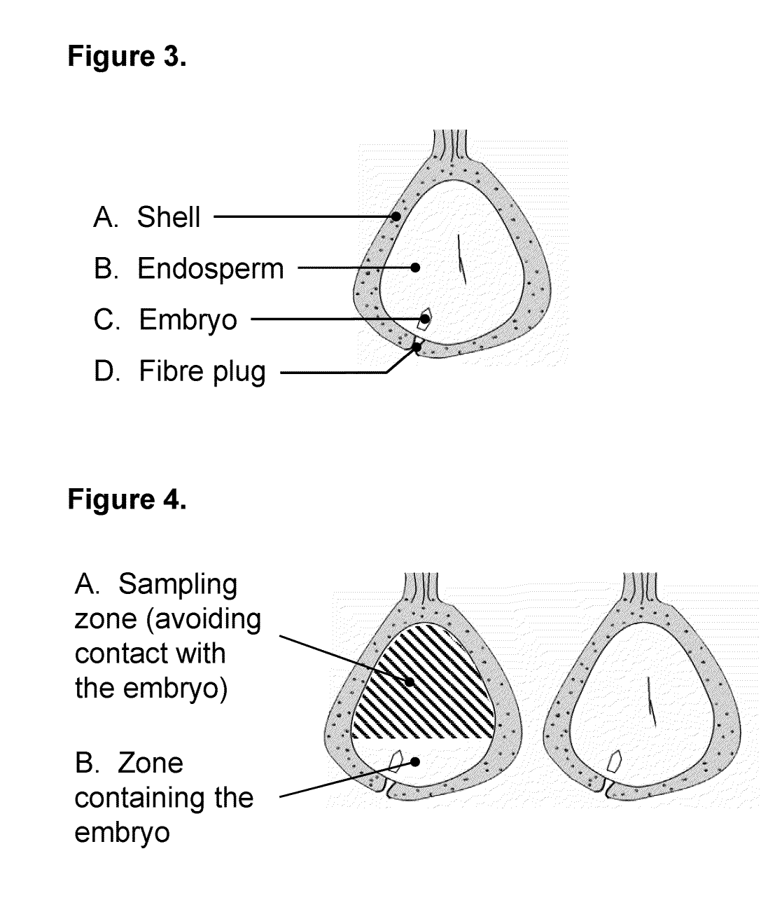

Following endonuclease treatment, size control DNA fragments (upper marker 15 bp, and lower marker 1,500 bp) were added to the reaction products, which were then resolved into 15, 100, 250, 350, and 1500 bp fragment sizes with an Agilent Bioanalyzer LabChip (P/N: G2938-90015) (FIG. 2-A).

Reaction products of DNA from dura palm samples yielded a 350 bp band in the ‘No enzyme’ lane, and a ˜250 bp and ˜100 bp band in each of the ‘ AcuI’ and ‘HindIII’ lanes (FIG. 2-B).

Reaction products of DNA from tenera palm samples of the sh^{MPOB}/sh^{DeliDura} genotype yielded a ˜350 bp band in each of the ‘No enzyme’ and ‘ AcuI’ lanes, and a ˜250 bp and ˜100 bp band in each of the ‘ AcuI’ and ‘HindIII’ lanes (FIG. 2-C).

Reaction products of DNA from tenera palm samples of the sh^{AVROS}/sh^{DeliDura} genotype yielded a ˜350 bp band in each of the ‘No enzyme’ and ‘HindIII’ lanes, and a ˜250 bp and ˜100 bp band in each of the ‘AcuI’ and ‘HindIII’ lanes (FIG. 2-D).

Reaction products of DNA from pisifera palm samples that are homozygous for the sh^{MPOB} allele (sh^{MPOB}/sh^{MPOB}) yielded a ˜350 bp band in each of the ‘No enzyme’ and ‘AcuI’ lanes, and a ˜250 bp and ˜100 bp band in the ‘HindIII’ lane (FIG. 2-E).

Reaction products of DNA from pisifera palm samples that are homozygous for the sh^{AVROS} allele (sh^{AVROS}/sh^{AVROS}) yielded a ˜350 bp band in each of the ‘No enzyme’ and ‘HindIII’ lanes, and a ˜250 bp and ˜100 bp band in the ‘AcuI’ lane (FIG. 2-F).

Reaction products of DNA from pisifera palm samples that are heterozygous sh^{MPOB}/sh^{AVROS} yield a ˜350 bp band in all three lanes and two bands of ˜250 bp and ˜100 bp in both the ‘AcuI’ and ‘HindIII’ lanes (FIG. 2-G).

All six assays reported the expected result relative to the known genotypes of the trees sampled (100% accuracy). PCR amplicons or other synthetic DNA molecules of known sequence can be included in the treatment and electrophoresis steps of the assay as internal (in the same reaction mixture) or external (in a different reaction mixture) controls to determine enzyme digestion efficiency.

1. A method for predicting a shell fruit form of an oil palm seed, or plant comprising:
digesting oil palm seed or plant nucleic acid comprising SEQ ID NO:4 by contacting the nucleic acid with an endonuclease that distinguishes between SHELL genotypes in a reaction mixture; and
determining the presence or absence of cleavage of the nucleic acid by the endonuclease, thereby predicting the shell fruit form of the seed or plant.

2. The method of claim 1, further comprising amplifying the oil palm seed or plant nucleic acid comprising SEQ ID NO:4.

3. The method of claim 2, wherein the amplifying generates an amplicon and the digesting comprises digesting the amplicon with the endonuclease.

4. The method of claim 2, wherein the digesting occurs before the amplifying.

5. The method of any one of claims 2-4, wherein the amplifying comprises polymerase chain reaction or isothermal amplification.

6. The method of claim 5, wherein the amplifying comprises isothermal amplification.

7. The method of claim 6, wherein the isothermal amplification is loop-mediated isothermal amplification (LAMP).

8. The method of claim 7, wherein the determining the presence or absence of cleavage of the oil palm plant nucleic acid comprises observing or measuring the turbidity, or color of the reaction mixture after loop-mediated isothermal amplification (LAMP).

9. The method of claim 2, wherein the amplifying comprises quantitative amplification.

10. The method of claim 2, wherein the amplifying comprises real-time quantitative amplification.

11. The method of claim 1, wherein the endonuclease cleaves a nucleic acid encoding a wild-type SHELL allele but does not cleave a nucleic acid encoding a mutant SHELL allele.

12. The method of claim 1, wherein the endonuclease cleaves a nucleic acid encoding a mutant SHELL allele but does not cleave a nucleic acid encoding a wild-type SHELL allele.

13. The method of claim 11 or 12, wherein the mutant SHELL allele is selected from the group consisting of an sh^{MPOB} allele and an sh^{AVROS} allele.

14. The method of claim 11 or 12, wherein the nucleic acid cleaved by the endonuclease is resistant to amplification.

15. The method of claim 1, wherein the endonuclease is Eco57I, AcuI, or an isoschizomer thereof.

16. The method of claim 15, wherein the endonuclease cleaves a nucleic acid encoding a wild-type SHELL allele but does not cleave a nucleic acid encoding a sh^{MPOB} SHELL allele.

17. The method of claim 1, wherein the endonuclease is HindIII, or an isoschizomer thereof.

18. The method of claim 17, wherein the endonuclease cleaves a nucleic acid encoding a wild-type SHELL allele but does not cleave a nucleic acid encoding a sh^{AVROS} SHELL allele.

19. The method of claim 1, wherein the digesting further comprises contacting the DNA with a second endonuclease.

20. The method of claim 19, wherein a portion of the nucleic acid is digested with the first endonuclease and cleavage of the nucleic acid by the first endonuclease is detected, and a portion of the nucleic acid is separately digested with the second endonuclease and cleavage of the nucleic acid by the second endonuclease is detected.

21. The method of claim 19, wherein the second endonuclease distinguishes between SHELL genotypes.

22. The method of claim 21, wherein the second endonuclease cleaves a nucleic acid encoding a wild-type SHELL allele but does not cleave a nucleic acid encoding a mutant SHELL allele.

23. The method of claim 21, wherein the second endonuclease cleaves a nucleic acid encoding a mutant SHELL allele but does not cleave a nucleic acid encoding a wild-type SHELL allele.

24. The method of claim 22, wherein the mutant SHELL allele is selected from the group consisting of an sh^{MPOB} allele and an sh^{AVROS} allele.

25. The method of claim 22, wherein the nucleic acid cleaved by the second endonuclease is resistant to amplification.

26. The method of claim 19, wherein:
the first endonuclease is HindIII or an isoschizomer thereof and the second endonuclease is Eco57I, AcuI, or an isoschizomer thereof; or
the first endonuclease is Eco57I, AcuI, or an isoschizomer thereof and the second endonuclease is HindIII or an isoschizomer thereof.

27. The method of claim 1, wherein the method further comprises sorting the seed or plant on the basis of the predicted shell fruit form.

28. The method of claim 27, wherein the seed or plant is sorted between predicted dura, tenera, and pisifera phenotypes.

29. The method of claim 27, wherein the sorting comprises selecting the seed or plant for cultivation, breeding, removal, or destruction on the basis of the predicted shell fruit form.

30. A kit comprising:
an oligonucleotide primer that primes the amplification of a nucleic acid comprising SEQ ID NO:4; and
an endonuclease that distinguishes between SHELL genotypes.

31. The kit of claim 30, wherein the oligonucleotide primer comprises SEQ ID NO:4 or a reverse complement thereof.

32. The kit of claim 30, wherein the oligonucleotide primer comprises or consists of SEQ ID NOs:9 or 10 or a reverse complement thereof.

33. The kit of claim 30 or 31, the kit further comprising a second oligonucleotide primer that hybridizes to an oil palm plant genome within about 8, 10, 15, 30, 50, 75, 100, 125, 150, 200, 300, 500, 750, or 1000 bp, or about 2, 2.5, 3, 5, 7.5, or 10 kb of the first oligonucleotide primer.

34. The kit of claim 33, wherein the second and first primer flank at least about 8, 10, 15, 30, 50, 75, 100, 125, 150, 200, 300, 500, 750, or 1000 bp, or about 2, 2.5, 3, 5, 7.5, or 10 kb of continuous nucleotides containing the SHELL allele.

35. The kit of claim 33, wherein the second primer comprises or consists of SEQ ID NOs:9, or 10 or a reverse complement thereof.

36. The kit of claim 30, wherein the endonuclease cleaves a nucleic acid encoding a wild-type SHELL allele but does not cleave a nucleic acid encoding a mutant SHELL allele.

37. The kit of claim 30, wherein the endonuclease cleaves a nucleic acid encoding a mutant SHELL allele but does not cleave a nucleic acid encoding a wild-type SHELL allele.

38. The kit of claim 36, wherein the mutant SHELL allele is selected from the group consisting of an sh^{MPOB} allele and an sh^{AVROS} allele.

39. The kit of claim 30, wherein the endonuclease is Eco57I, AcuI, or an isoschizomer thereof.

40. The kit of claim 39, wherein the kit further comprises a second endonuclease.

41. The kit of claim 40, wherein the second endonuclease is HindIII or an isoschizomer thereof.

42. The kit of claim 30, wherein the endonuclease is HindIII, or an isoschizomer thereof.

43. The kit of claim 42, wherein the kit further comprises a second endonuclease.

44. The kit of claim 43, wherein the second endonuclease is Eco57I, AcuI, or an isoschizomer thereof.

45. The kit of claim 30, wherein the kit further comprises a control polynucleotide.

46. The kit of claim 45, wherein the control polynucleotide comprises a DNA sample containing Sh^{DeliDura}, sh^{MPOB}, or sh^{AVROS} nucleic acid.


SEQUENCE LISTING (13 sequences)

SEQ ID NO: 1 - DNA, 13 bp, Artificial Sequence
  synthetic portion of Elaeis guineensis oil palm wild-type DeliDura-Sh SHELL allele polymorphic region
ctgaagaaagctt

SEQ ID NO: 2 - DNA, 13 bp, Artificial Sequence
  synthetic portion of Elaeis guineensis oil palm mutant MPOB-sh SHELL allele polymorphic region
ccgaagaaagctt

SEQ ID NO: 3 - DNA, 13 bp, Artificial Sequence
  synthetic portion of Elaeis guineensis oil palm mutant AVROS-sh SHELL allele polymorphic region
ctgaagaatgctt

SEQ ID NO: 4 - DNA, 13 bp, Artificial Sequence
  synthetic portion of Elaeis guineensis oil palm consensus sequence of SHELL allele polymorphic region
cygaagaawgctt

SEQ ID NO: 5 - DNA, 733 bp, Elaeis guineensis
  oil palm SHELL coding sequence, wild-type SHELL allele, DeliDura allele, DeliDura-Sh, Sh+, EG4N37875
atgggtagaggaaagattgagatcaagaggatcgagaacaccacaagccggcaggtcactttctgcaaacgccgaaatggactgctgaagaaagcttatgagttgtctgtcctttgtgatgctgaggttgcccttattgtcttctccagccggggccgcctctatgagtacgccaataacagcataagatcaacaattgataggtacaagaaggcatgtgccaacagttcaaactcaggtgccaccatagagattaattctcaacaatactatcagcaggaatcagcaaagttgcgccaccagatacagattttacaaaatgcaaacaggcacttaatgggtgaagctttgagcactctgactgtaaaggagctcaagcaactcgaaaacagacttgaaagaggtatcacacggatcagatcgaagaagcatgagctgttgtttgcagagatcgagtatatgcagaaaagggaagtagaactccaaaatgacaatatgtacctcagagctaagatagcagagaatgagcgagcacagcaagcaggtattgtgccggcagggcctgattttgatgctcttccaacgtttgataccagaaactattaccatgtcaatatgctggaggcagcacaacactattcacaccatcaagaccagacaacccttcatcttggatatgaaatgaaagctgatccagctgcaaaaaatttactttaagtatgtcgctgcttgt

SEQ ID NO: 6 - DNA, 733 bp, Elaeis guineensis
  oil palm mutant SHELL coding sequence, MPOB SHELL allele, MPOB-sh, sh-
atgggtagaggaaagattgagatcaagaggatcgagaacaccacaagccggcaggtcactttctgcaaacgccgaaatggactgccgaagaaagcttatgagttgtctgtcctttgtgatgctgaggttgcccttattgtcttctccagccggggccgcctctatgagtacgccaataacagcataagatcaacaattgataggtacaagaaggcatgtgccaacagttcaaactcaggtgccaccatagagattaattctcaacaatactatcagcaggaatcagcaaagttgcgccaccagatacagattttacaaaatgcaaacaggcacttaatgggtgaagctttgagcactctgactgtaaaggagctcaagcaactcgaaaacagacttgaaagaggtatcacacggatcagatcgaagaagcatgagctgttgtttgcagagatcgagtatatgcagaaaagggaagtagaactccaaaatgacaatatgtacctcagagctaagatagcagagaatgagcgagcacagcaagcaggtattgtgccggcagggcctgattttgatgctcttccaacgtttgataccagaaactattaccatgtcaatatgctggaggcagcacaacactattcacaccatcaagaccagacaacccttcatcttggatatgaaatgaaagctgatccagctgcaaaaaatttactttaagtatgtcgctgcttgt

SEQ ID NO: 7 - DNA, 733 bp, Elaeis guineensis
  oil palm mutant SHELL coding sequence, AVROS SHELL allele, AVROS-sh, sh-
atgggtagaggaaagattgagatcaagaggatcgagaacaccacaagccggcaggtcactttctgcaaacgccgaaatggactgctgaagaatgcttatgagttgtctgtcctttgtgatgctgaggttgcccttattgtcttctccagccggggccgcctctatgagtacgccaataacagcataagatcaacaattgataggtacaagaaggcatgtgccaacagttcaaactcaggtgccaccatagagattaattctcaacaatactatcagcaggaatcagcaaagttgcgccaccagatacagattttacaaaatgcaaacaggcacttaatgggtgaagctttgagcactctgactgtaaaggagctcaagcaactcgaaaacagacttgaaagaggtatcacacggatcagatcgaagaagcatgagctgttgtttgcagagatcgagtatatgcagaaaagggaagtagaactccaaaatgacaatatgtacctcagagctaagatagcagagaatgagcgagcacagcaagcaggtattgtgccggcagggcctgattttgatgctcttccaacgtttgataccagaaactattaccatgtcaatatgctggaggcagcacaacactattcacaccatcaagaccagacaacccttcatcttggatatgaaatgaaagctgatccagctgcaaaaaatttactttaagtatgtcgctgcttgt

SEQ ID NO: 8 - DNA, 27001 bp, Elaeis guineensis
  oil palm SHELL genomic interval
gttggtcagctgacctctaacaagaaagactattcacatggagggatgacccactgatgccccaaaacaataatgcaaacaaagagagggtcgctctctcacttgagcagcgtagggatgccagtgagtgcaataaagaagtggggacgaggtattagaatttcgatacatgtgtgcgtgtgtgagtatcacagagagagagagagagagagagagagagagagattgcatgaaagtcctcagagtatgggacatctccaaaaccaagtccaatatctagtgatgggctcttttatacaaagagtgatgcgcaagaaatagaagacatggaggtgagaagcttgatccatgcatgcatgaacatgatgtgagagagaccatgaagctgaagaaaggtccatagccacagaggcaataaaagaacatggttgggatgttaaatcacagtaaatggtgaaaagaacatggtgcaactataaggggaactagttttagtagttcatctttttaggaccacaccgcaaggtggacagttggtgttacatttagtctctcttcattctcttttaagggaaaatgtcatctagagtttgcagaagttttgaagttttacaatatcgcttaatttaattcaattgatgaacaataatatttagtgattgaaggtgtgaactgtaaggtcacttgaaatttagagtctatacacattgggagttcaaaatatctggtcaattttattaaaatgtattgttccaatattaaaattttctcgagttttctttaaaggactctagtgttcctttgatctaaaaacagtctaattttttgctaccacaaatatactacagtagaggcaaaaaaatctagataacacaaaggaacaaacaactttatgtttttaagcaagcaaataagtacatattcttccaacgttttctccaagaacatgatcaatagttcaaaatgtttgtcccttgatattctttacagaaaaaactcacggacaataagcttaaatcttcatggccagtatattttgtaatatatggtgaaactggagttcagttgttctacaatcctaataagatcataggggtgcaattcttgtgtccttacacttagggaaaaagctttatgccccagctagaaagattatatcgatggtcccggaggagtcttgatttagtacataacttctaaatgtggagcatcgcccaggaaggaaatatatccatattaacaaagtttgcaacatttggattggatgatagtccaatgaagaaaaattgacctactcaaccatgacaatggagctgtcctcctaacatgataaggacatagcaaccatactttggtgacattttaaaatcatgcaattacttcatcatgtttaccatggaaaattacacaagaagatggaaaacatagcatagcatttaccataaagaaccatgcatcgttacgtcatccaagcgattcaggtgcatgcatgtagatttttccnctctttgatctatatatatatatatatatatatatatatatatatatatatatatatatactaannnnnnnnnnnnnnnnnnnntatctaacttaataacgaagtcttatgtatgctaagttttccctttagaatacttggaaggcttgacagcatgcaatcattcaatcaaaacaagagagataatagattgtttcagagctaggcttttggaacaagtaaatatagtaatacaaatagacaatacaaaagacaacatttttgtttccagaaaaccattatagaattttctaagcctttcccttctaatgaggaacatgttcattgcaatgttagagctatatgactaacttgtataacggacatgccacgttagccttgagtgatcttgaaattgttgaattagtccaacaaatacgactatttgctgacgcctagcccaaataaaacagaattaacatgcagtctagtctgaattagtggcttccactaaggattagaatgtttctcctaactatagagcaagaagacctcttgtatttagcaatgttatccaagctggtcgttattattcctctagaattggctcccactgatcagctaccaaagttagtccacagcacatagtcccaaacatgtcattaggtttaccataaatttagtatttcttggtaaatagaggcaaacttgctgactgttcatgcaattaagttaccttgacatatgtgatgcattaattacatgcaaagaaaccatctatcatttggagcttatactggaaattgaatttcccgagagaatctatatattcatattcgttcatccctcgattgtattttcaagaaaagatttgaattttaggcaacatacctttttctctattggtcttaattttttacatttccaactacttaatgatatgattcagatacatagggtttctacccatttttcttgaaaagaaaagaaggaaaggaaaatccaatagaatttacagtgcattgttgaatctgcctaatctgaaccatgactttaaaaaaaaaaaaaaagaagaagaagaagtttctggctacatttttatatcagcttataattagtttttattaatagtactcaccaggaaaagtgtaaatattaattgttcttatgtattattgcttttcttagtgtttttctgttaggaaaacaaaagaaacttgaaattgcactaactacaagtataaaaatgaaaaaaggtaaggccaatgttagagagcactaaggcttcatggtataaccttcaaaagttgtgatggatacaataccataggtatttgttcttgctagccataaaaatatacaagcatatgagggtagcacatggtaagtagacacaaggtatatttatcttcatgatatcactacaataacctgatgatatggtgatgatgttatccataaccttatcaataatttggttttgattttctacaaacagggatgtgtttttattctttttgaatatatatatatatatatatatatatatataagaaaaaaaaccttggcaccggtagggcatcatgttaattaacagacatgataatgtagtactaaaagctacatctaatataattgtacttttactaacttgatgtgaaacttaaaattgtattgcttgggatcaccactttatgttgcaccacttttgtgtgttcttgagcatatctgcgcatatcctctttcactcgcattatttagatattaagtgcaaacatttaagaaccagttatccttgaaagacatttgagactgcatgaatgccatcagatttcttggccaatggaatgaagcaggaatggatgtaatgtgatgtacgtgggtagaagggacagcgttaacatgaaacatgtggaagttttaatattttgagaagagatacatgcaagttacaaaaaaattaaaaagaacatacaaaaaaaaactatttatagatgaaagaaatgaaaagaaagaaaaactttaaaagaaagaaaaaaactttaaaatacaccgcaacgattaatttcttattcaccatgtcacctcttctatgaataacgaatcaagaaaagatctgtggcaggtattgccaagccaagcttcaataaggttttgttacttctagaatccaaactcccttgacctcatagttatgcgagagtctagtctattcattacttaattcccccacctaaccctaatctgagaaggaattattatggcttggggagatcacgtgcggaggaggcgccaccgatcgttttcactttaccgcgcgcttttcagtcctagcagtgcccccccagtccccaccacttccctgtcaactgctccaccgcaacctctcaatctcctaacaatcactccaataacgacaaaatgatggagctaacccatgacattttatatcgaagatatagtagcacaaactccatccggcggtacagcttacccagccaacccaccctccactctttaagaggaaacccacatatccgatgccaattgcatgcagtggaaagagagagagagagagagcgctgagctcccttcttttccctcggaatttgcggtgttggacttcaccttctcttccggcgtggaagctgagttcccggagtggtagttttttcctttttttttttttttttttgagaacaatttctgtctttctttctttctttgttgtttcggctttttgatatttgtttcctttcttgaggaatagtttttatgataataccggaattgggggttgctagaaatcgtgaggtgtcattgttataggttttttttttttgctggatcttcggcgatctttttgctgatattatccgtttttaacgatcggaacgttggttgcgtcaccaacaaaatgcgactttgttctcgcttctcgctagatttgctccacgaaaacagtagctttgtcggatcggatcgtttgctccatgtctagtttttagggttatcttttcacatgttcttgaatatatctttgtcaagaaaaaaaactttccttttgttctataacaaaaatcttttcttccacgtattttggttgccctagttttttcttttttttgtttgtttgtatttgttagagaatcgagatttgagccgtacatctcgttcataaactgtttcatttgtcagaatttaaagaaactaaacttcacgtatgcatcgttatccatttgtcatgatcttgtctcatgaatatatgtattctgttcttgtcttcgccttaatttttttctcactttcttttccgaaccctaacccattcaaagttcccaccttttctctcttacttgctttcattgttttttttccttttttttttttgttgcttttaattttgcttgaataccttttgatctatggaaattaataagtcaatatgtcagtatgtgaaggtctaggccatgttagtcccatcatttcatttatagtttagatgatgattttttctttgttcttggcaatattctagaccaacttcagcagacagaggtgaaagagagatcatgggtagaggaaagattgagatcaagaggatcgagaacaccacaagccggcaggtcactttctgcaaacgccgaaatggactgcctgaagaaatgcttatgagttgtctgtcctttgtgatgctgaggttgcccttattgtcttctccagccggggccgcctctatgagtacgccaataacaggtatgctttgatgacgccttctcttccttcgctcatatcaagttaattttatggcttcatttgttctatggccaagccaaattctttttaaagttctagaatgttaatgatggtagttttgctcctcttcaatttatttgcttcccttttatccctgatccaaatggttttttcttatttaaaattaccctttcaaattatcacatttaattcagcttttattattattattattgctatgagattagtttgttgttaagattctatataggaaggaatgatcaagtgatcattactttatgtagtaagaaattaacaagcaaaagcagcgtgcttggtcttaccatgaggatagaggaagagttttaccttgactagggcaactaaaaagggtttgagttttgcctaacattcttgttatatgaattcgatctgtacacgacttactactctggtattagaacgtatgtactaagaagttttcttgggatggaaagaaagaaatagtgaagtaaatcttattaatttgtctttacatgttcttctcatattttctcatactcttttcttatgttcagacttacaccgaaaaattaagatggatgatattatgttcttggtataggatttcttttccgacgtaccaatgttttgttaaggaaactttgtagttgacttttcgattataatattttccaaaggactccacgaaatggtaacatctccagtcgtttcaataaacttccgatcatattatgggtttgcatttaaggttttcttcttcccttcgcttcctctttgcctttgcctttctcgtgttacacttctggctcgtcaatctgcagtactctctagccatctctctctctctccatccacaccccccccccccccccaaacaacacacacaccctcccaccctgtgcaactaattccgacaaaataagggtctcccattgttagagcacctcctgtatggactgcattctaaagatacgggaaaatgagccagatatctattcatcaatcatttagtagagtctctaaggttcctttttaaaactgtcttgaaccggcttttgcacaaagagcaccactccctttatttataagttgaaccttcctgaaagctaacttgggtttcagctttcactgttcaagtaagtcataaagtttttctggtcattaaaccttgtgatatggagatggaaactgcttttctccctgccggatttctcttctggtgctaagcggctaaaacccatcatccatgtctgccttcctcttttatgggtagttgctggacttcggacaccggtgaaggatgaaggctctttgtgattggttatgagatatttcttgggccccgtgcctttgctgtcattccccacaactcggtggccatcatagcttttttgttagctttcgtccttacaaattcctctctggttctttctttcaccttttcatgcatttagcttcattgtcaacatcaagggaacacagaccggaagaagagcaagaagaacccaatcaagactggattttacaaatcaccaaaaaaaaaaaaaagactggattttactcgacgacgctgcagtcttctctgctctcactcaattaaaataggaacaaggaaaatgtagatattttttcccttttcgttatgataattataatctaagaaagattttaaaagcttactgtaatcatctcatgttcaactattgtgttgcacccaacgaaatttctgtgtgcctcatgacgagcattacctttccatggttctgacacgacattgcatactagattttactgtgtacgtaaaaaagcactgcatgacatcatctctattcttcttcttcatccctttttttttttttggtaatatctattcttctttaatgtcctctctgataactggtcttatgtacaggtacatctttacaagccttccgagagaatatgaatgcgtattttctgcaaccgagtatattctacaaataaaatgttagagattgtctcgaactggtatacaagcacattcctgtacgtgttgacatgaaagaagcatagatcagacaaaaataatagtacgtgacaaagatcataaagggactacaacattaggagggctacaattaataataacaacgggagcaactagcagtgtatacggttcagaccagatcagtttagagttatttttggaaccgagctgacttagttgtttttctaaagcctaaattgaaagagaaaccgattcaacctgatccgaaatactcgatttggtttcattcagtttatttgggttgatgttccatagtttgggctacattgattgcactaagtctaaatataaaaatttcattttatatagaattgatgttcaatggaccggactaagtttaaacatataaaaatatttaaattttgaaatatatatgtacatatacacatgtaggtcgggtaggtttaaatatagatacttacatataaatggattgggtctggttgggtcaatgcgagtctgcttaaatatatatatacatatgggctaggtcaaatcaggttagtttatatatatatatatatatgtgtatgtgtgtgtatcggatttgttggtttgcactgattttctaaaaaataaaatcgaaatcgaatcaaattttttctgttcaagagtatctcaaactaatttttaaaaaaaaaatagatataactagatcaaaaaagaccgatttgaataggcactttatgattttgtcacttttttgtatacctctacgagcaacaataatagaaacaataataacaattgtaataatcaatataaaaattattaatagtaattaaaaaatatatacaaaatttttgcaagcattcaagtggacacaaatcaaagggcactaatgcattcataaaaggttgctagctttgatccatggtagcatcgtgtatcctgaatcatttgtaggttaattgaaacctcctaaattgcattttaaataaaaatagcatgcattttatgaagataaccttactattattaggtaataaggttgttaaattctaacctaattaatatctcctaattaagagataatagttgtggtggaaacgagatggagagaaagcacttcatctctcatctttcctctctaaataacatttcactaagagatttaattattggatcaaggctagccagtttgttatctttttttgttctagaagtgttgaccttttaccatttctcatatgaaaaaaaacattatctaagaagaataaattattatgatcatgaggagagagaaggggtgttaaggttattggattaaaatcaaactgattagaggaccttgtttgctattataaacccatgacaacaatcaatatgcactaaagttttggtataataagttatcatcaagggccaaaaaggattaactcgagaacacaagcatacaatttaacatcgtacatagataaattcaatttaggaggtaattattgatgtatatgcttagacaagaagaaagtatagaaagtaaggaaaattgcttgaatgatttagaagtgcataaatataatctaaatttgaggagtcttatttatattttgagaattagaaagggggagaaaattgaacaaaactttgataaattgattgtaagaattttcaaaccttaaagcgaaagtgatagtgaagtggaaccattaaccataatttgatattaaaaggttatctatattcattaatatcatttttttaaattattgtatatattttttgggaggaaaattatgttctttaaaagttagattggtttcgtacttatcataaatgataaatgatctgaatttagtttgaaaattatttgacatataacctttgataagaaattacatattaggattttatggtttactgtcatatagttctttgttttgtatttatttaactttgaccataaatgatttgaagtcaagatactttaattctagcctccccatcaacatgttgatggcttatgatttttattttttataacatttatttttatctataatatttttcattattaatttaatgaaagtggattacacgacatctaacttatatttttgaaaatagagaatgatatggtatactattttgaacatgttcagaaattagaatcttattattgttttcataaatttaaaatattatacttgtcaatatgaaacatattaaaaatgatctaaaatattttttaaaatttaaatgtcataggtttgaaaaattcttacttgtaaatatatgatttgtaactaaatatttttaatggcatgatattattttctaattccatcaaagatctagaacctttcaaattagttgaacttagatacgtgttttaatatgtcttaatcaagatcaacaatttgacttcttattttatatgatatatggtatattctcacttgatgcatctgtaagaaaatttaaaattcttttattttattttatatatgtgtcaatactataattcttgattatatggatttctagtacacttagattgtgattgttgtttggcattggaagatcgagacaattaataccataagtgggatatatacttgcttaacccaacattaaaccaaaatcacctctcgacaatcacacaaggacaagtgtactcaagataaacatggattgtcaagatatatataagaataataaatcatgtatataattttttttcatctacaaacttctcttctttctcttaatttagtatataacttccataccaatagattactttttatttgacccaaaaatcaatttaaccttttgtttttaatttaatcctttgtcttcttaaatgactcagcttgtaatttaaatatatcattacttatgcagtaatgtccatttgttgtaaataaatatttggcaatagaagaaatactataaggtcaaaaaatataaacaggacaatgaagcatttcgtatagtctattataaatgtagaatgaaacataataggcttttcatttgatctataattaaatataattatcaaatatcaaaattaatgtaccaatttatgagacatcaatattataaagggatactaagaaactaacgagagtacactgatcaagaaatgtgatcgaccagtgaggcatgttgtttataaattaattagacacgttttcaatttatgagaatttgtattcaaatatttataattgataaatggatcactttttatttgctctttatatttagactccaaatagtgctgtaagagaaaaaaatttgaaaaaataatttatattttcaaaaaaaatatttttatcaattttcattaccataaaaaaagatcgaaaataaaattaagaaagaatgagataagtttgataattttagacatccgcttgcataatagttcatgtttaatattaatttatcactgagaatgcaaaaatatataaaatttttaattaagctttgctacatataattattatatatcacttacataaatgatttgattaaatatttttaaaattttaatctaatttttaattgcatagatatctattgcagtaattttcctctaaataataaattaaactaagaaaataaaaaatatttaataacaattggaatatacttatcaatcctataagaataattcctgtaaaactccatccatttaacttgcatcatgcatcatttatttttttatttttaatcatttagaaatttaaaatcaaaattcaacttttatagatttattagaatgcatggaatgatttcataaatgttgcattgtactaaaagaatttatgataatctatacaagtcaagcatttaaattacttattatactttacaatagtaaactaccttttgagcaaaatggtggtaacccatcataatatgtcatatgataaataaaatgaagtacaaggctagataaagaagggggacagaaagagagatatagggctcccgagcttaagcaaccaagcaattcaatatagttgcaaccaataaaatccgatatgagcaaatataaatataactcactaaaagcccaaaatacacccaataagcccaggtaaccactagcccaaacataatggcctactaagtttgaattttaaagtttaagtctttgcccattcaccacctctaccttccaagctctaattattttaatgccattgcaaatcatcatgtcttctcttcttctaatttggtgtactatcattttcaatgtattgcaactctattgccaatcgatgctcctccaaggcatctctcaagatctcatttggctctattgcgctaccactaccatccatcctattactctctagatcaatatcaaaattaatgcagctcttttctcgatactttttgcatcatcatctcactccatcatcacagtctatacttgttatttggagcctatgcttgctactatattgtgacctcaatattacaatctctacttgttaccactcaatgtccatgcccatcttccatgaaagctacttggcaaacaatcctaataggttccaaaaaaccccacagctaaggactaaacacacaaccacacaggttctcttatactctctttatttagattcaaatctacatactaggctaagagtccaattctagtcaaactgaatcataaacatcataatcaatccatggtcaactccaaccatttatgaagcataagatcttatctaaaaaaaatcaaccaaaagttattatttggcttccttatccctatataagtatctaaatatcctttgtgtacaaccaatatcaaataaaatacatgcttgtgcggagatcctcacaaaaatatttagcaaaataatcctatcaatgttgttaggattgatgttgagtcataaatcatatatatagtggattggattagattcatcatcgatcgaattatagatcattagatctttttgaattatttaagatttttaaaatatacaagaaatgcaaaaattaaaagtataagatgaatatagaattgatgaataaagaaccaaaaaatatactgaactaaaagagagagtatgttggcttaaccaattgtagcaaggtagaaaccccactatggtatgtataacataagaaagaccgttattaaaagagaaacagatgcaaatcactttattctaagattaaaaatactatcttagctagctactttaaggatacaacttttacacattcactcacaaatcctagagatttagcaaaagagaaaagagagaaaaaagaggaaaggcaagggaagaatagttcctataatagaattttcactagttaataactcaagtatctctaggatgactacaagagtagctccaataggatatttgtaggtaatatataggaactcaattcttaaacttttcaatgtgggattccaaatttcattctaactccaataattgtaatgcaaatttttctaacaacatcaaaagttattaacaataaactcataacaattaaaacttttcaaaatttattaatatcaaactctcctaacttagacaaaaaatggatagaaaaataagtaatcataaaaaaactagttgacccaagtttataggatttgagacctatgtagcctaaacctataacccatgtggttagaacccacgatctatgttgttaggacttgcaacccgctctagcatcacatacatttaagaaatctaatttgcttccttataagttcatatatatggaatgtctaataaacaaatattgtgcctgattattcaagatctatatttgtaccatctgccaattaattatcattttatttagaatgtggttaaaaaatataaaaatttctcttttaagtccataaactccaatactatattagttaccttactaaccaagatctagaaataatttaaaatctataaaatttaatgaattatagaaattggactacataatccataatatgacattaaattctaatttcttaatagataatgattcaaagataaggtccggattgtttatggccattttatctagattgtaagatgcataacttgaatgataagattttaacaacaatagctcctattaaaaattaaaaaaatatttcttatatagttatcataaaaggtggtaatcaagtcatattatatttatcaaagcactgtctaagcaatagctacatgatactctatagtatctaagcactattctcattatctttatttctctttttaaaatttagtgagatggttgcattgcctccatctatgacttaaatttttggataacaaagccatatctattaagtttctttaatgaacatattttggctcaagtccattaggataaaaatcttttagagagcatgaaaattatatggttagaaaatgttactaaaggtgatttcatatgattcttaatgtctaaaatagtgtttaactttcttttctctatttttagtaaccaatgtcaacaaccttaatgaagcacttgaaaagatcgtctccttaataatttatattgtaagtttaaattttagttccttgaactattgataactaattgttacttcagtaactcatcaaactatttttaataattctctccatgattctcttacatgtccttttaaaatgcaacatgatatatcaatatgcttttctattagacaattcaacttctaattatgagtaattaaatatataatttttattaaaatggatctaatttttttggtttggcaactcttttgttcagcataagatcaacaattgataggtacaagaaggcatgtgccaacagttcaaactcaggtgccaccatagagattaattctcaagtaagaaagacatggcaatttaatctaaaatagatttctctgaagtccatatatttttgcctcatatgcttatcagttaaaattcttcatgctcataaaggcataaaagcaagtcagtaaattatttgtacagttgatctttttgttgtttgttcatagccttacatgtatctttgaatattttgttgatatattgattgcacagattttttttcttatttccattgattgttgcttttcttggatatatttgataggtttgattgagaatagtggattaaggtggtttacaacctttctttgagagttgtaagggtgtaaagggctagatctactaagagatgagggtgatgatactactaactattaagactatgtcggagtcctttttcttatggataacatatatttgaagttgtcattccttataatgtaagaggtaatgaaaagatttttcttgcaaattagaatcactttgcatcaactccaatacttttctttatgctaataaggtagtgaattttagtgatatcgtctaggaatgattcaactaatacctcattgcttttgaaccataattgcttttcctctattttcttttttttcatttcaatcatatttgttatggtgtaggagggaaggtatcattaatcccatattagttgtgagtcaaggaggactctgaaggaactacccatcctacatgaggtgctttttggattgaaatccaaagggataaaattttgagacctatgagccaaaacggacaatacgtaatagccgagccatgagctcttggttgcaacagtggcacactaggaaaggaatcacccttatgactgtgggatccctctcatccgtacacaaactccttgactggagggcggtaataaaataaagatgatggtaccttccgtctatccatagacttcttcttgactgggggctatattgtggcgtaggaaggagggcatcattagtcccacattaattatgaattaaaaggctgggactctaacttatataggaaggaaacactcatcctacataaggtgtcttttggattgaaattcaaagagacaatcatgaggcccatgggcaaaaacggacaatacttcacatgccaagccatgagctcttaatcataatagtggagggagggcatcattagtctcatattggttgtaaatcaaagaagatcctagcttatatgggatgggaccttttcttctctgtgacgccccatgccatgtcaatcaaagatcgaagtggataatacctcacatatacaagaacggagatcaatctacccgagccatgagcccttgactgcaatattgatgtgccagataagggacctcccctacgactgtggggcctatccctctgtacatagactcttccttgattatagagctatattatggtgtaagagggaggacatcattaatcttacataggttgtgagttaagagagactctgatttatatgaaatggaatcttctctcctctgtgaggtcccatatcatagtggctaaagatcgaaacgaataatacatcacatatgcgggagtgcagactgacccatctgagccataggctaacaatattatcatagaatcatgatttgtagtgagcattcatctactattttttttccagaactcatttcaatttatctcaattttcatgttttaaaagaaggatagatcttgcccaataatacatataatatttatgaaagtcctatgaaagccttattgtagtcagaaaacaaggtcaaaaatacattctagtctatggttgagaacttcaaccaattgactacgttgcctgaatgttcaaaagaattcaatagttcaatcaacaaatagagatgggatcatatcatctttttatttcatcaattttgctgaatgatatctataatatatatgtgccttcgctctaaaaatctttggcctaagttcagatttatgatagcaactcttcaaaaaaccaaaaattgtgatgacaactgttgcctaggcgacatgtaagtggttaagattgaaaactctaaaatagagtgcagctactctaggtaaaagatcactgacatagacatacatcaaagttcgtctgctccttaattatttcttttactaaagtagatgttgaatcataggcgaacaatactactgaacaatacatatttcttgcatacattggcttgactattagtgctatgcactctaggttcatttatacttcacaagagtttttatttgtttgccaacatcaaatttcatgcaatcaaaaacacaacttgcagaaaaaatgaataagagttaagtaaaaggacctaattatcataagctatggaagacaagacaagggatactgccattgatactcttagtaaaatagtgttataagtgatagtaatagcaatatgaagaaaagtatgaaagaactagttttttcttaaaagagtatgaaatgcatacaagttgcggtatcatttgtgaaagagaaagtattttcttttatttacgtttagtcaaaaccatatttatttgttatagctgatccctgaaatttcataactcacaccaattggcatgatgttattagcttagatttgccattataccgccattggtaagaacaaaatgccctcaataccaaaataaactgcatttgcaagttatttgaaagaagtgcaactctattattgtggcatgttaacgagcttttctataatttgaattttttgtaccgtctatgatggttatcaaattgtataacgaaggcaaaaaactatggctaaatgattcgtttttatgaattattgtatgctactcatgctatactttgtttgtttcatcatgatcattacctgcaaatcatctactcgcatgatacagctacgcatttgtcacattccgaacctagcatctgggtcagccatgcgatggcctcatattccctaaggcaaggctctaaagaatatacaaagtcttaacttctttacatcctcaaaatcacatgagcactattgatatcaatttaaaataaatatttttaattaataaataccaaactttatacaatttatagaactcaagtatctgattggtgttgataatgctccatctaattaattccttcaatcttgatctcaactataaccaaaaatacatatatcctatgactcttaaaaagaaaaggaaaagaggagatgtaagctttacaatccaataagaattttcacacaccaacactaatataataataataaataaaaaataagtaatagttgttactcttataagtcttctgtcacaagataccataataataaaacatgcaaagaaagtacatctttttatataataaatcatattaaatacttttctcaatttatagttactggattcgataatatttaggtactcgacactgaactatcacaatcatgtttctggctcatggcaagatatcgacatagtggctagtctcctaaactatcacaatcatatttttgatcagtggtaggatatcaacatgatggctaatctagagggccatatgtgcttaactctgactactacagctagattttcaatctatgatcagacactaataaagtagcttatctaaaggattataagtgctcaattctaaactatcataatcacattttcagattacggtaaggatcaatatagtggcttatcacacagactaccacaactatatttccagcttgtggtggggcatcaacaatattaatagcctaaagcaccacaacccactattcagttacacaattcaattaacaattcaagatcatactcttttgtaaaattcaatacacaagattatcgaaactactcaagttaggattagtgatatatgatacataacttcatacagatctctctctatatatatgtatgtatatatgtatgtgtgtttgtatacatacaattaacaaataagcacatagcatgcaactatcaaaaatcatataaatcaaaatcactatgcacaaaataattgttatataaatagatgatcaatgtctagaaatttttcgctctacgtactgatgacaaacttggttataactatttattagtccatgcctagcatgtccaatcaatcaatataatttcagttcatcctgaatcaaccattagcataaaaattaaataacttattgccagtgttttaatttcaaagctctaatattcgaattaatcttacttttaatttctctatactttctaattaaaaattacataattaaataaaaaatttggattttatcttgactataaactagagcataaaatcccttgctcagtatttcttaattggatgattggtcccatccaaaatcaaaatcaataattagaaagagccataaaatggttatcatcgatggtagaaaatttagaagaagaatatagctagtcgaataatagcatctggcaacctaagtgggtcaggtctggctgatttagctggttattcatgaatacaacaaagatcatctatgatagattttgaggttgcttgatcaagaccaaggtggagaaaggcaatgctagtagcatccagtgactagtgttagcccaatatacataatccaaatgattagtctacaaatcaaaagccaaattatagctcaataggaatttggtgatgataaaatcaaacaatgcttagcagggtcgggtttggagttaactattatacagaacaagtccaaaaccttcttctaggatcaacatagggtctactatcaagtcttcaaaatccatctttttttcttcttttttatccctaaatataagaagagagagaaaaagatagaggaagaaagctggtagaaagagatgtgagagagaagggagaaaaaaagaacaaaccaaatctctctctttctctcttcattatcttctttcttcaaaaaggggattttcccctttctccctctctttctcctagcggatggctaaggccagcagtctatggtggtgtctagtggtgaagatgcgatggccgcagtgctacagcgatggcagttgtcggttggtagtggtagtgggcgatccaatagaaagaaaaggaaatcaaaacaaaatagaaaaagaagccttgctttgatatgataagaagactacaaggtggtcagcggtaatgacaagcatccgcattagcttaacctcttatgatagttgcaaaggcaataatagccacagtggctgacggtggataaactaaagaaaacagaaggtccttagtgcggcaacatccgacggtcttccaagaagatttcagccaatgatggctgtatgaaaataaaaaagaaaaaaaggtaccaataagatgggaggccacgatgcctcgatcatctctaatgatggcgtgaccatggttgtgccaaaacttttttttcctatctttccagtgatatgatcaattgatcactgatcaatacagtggaagcttggctttataaatgatggaggctagggttttctagcctccgatgagttggagaaggagccagagtcaaactctttcttcgactcaaataggggaagggaaggtctttccttccttcccattgtttcttgggcttgatgagatttttattttagaaaaatttcaagcctctacaaccatatgaaatcataatgtcaaagctagaaaaggagatctatgccataatattccaattccaagcctaatcaaagaaccatcaatccattaacactaactttaagatacctaagttctccctagcattatctatggtaagaaaatctattaattaaaattgcataattatatctaaatcagtcaaagaacaaataatattctctctttctttatcaaaattatactcctttaccaggaaactaattcgaatcttccataatatcttttggatcaaagaattaatgtatttaattagtttcaaaataactcaaaccatcacacttctgctatacactctaatctaaatccatcgattcctctgggttgactaggtgaattctaacaaaataccgcttaatatcggaaccaagaagatccaaaattttaacttaaggcaaactagaacaaaacttttgcatctttttatccttacaaaatcttgagcataccacatcaaaagtaaaccttgagccactatccatgtttgaagcatgacataagcttcgccatcctctcaaaacttaataactctagataaatttaaattaatcctgacttctctaacagttcaattagactatcaagatcaccttttctcttggaaagttaactcaaaattctaacaagttagaaaactctaaatcaacattcttattaacttgtttattttttatagagcttcgttcactacaattcttagtatcaatcgacaaaccacatgaacgccccttatatgttagacatatacaagtccaccagaatcaatttctctactcaattaaatatcgatagcaagatactatagacctgctcataagcctaactctgattagaatttaacacatccaactatctccaacaaatataagaaagaccaagtaagctgatctaaagatgataatttaaattatcaaagattctaccaagatgcatatctcatatccaattgataaaatctaatccattaatagaatcaaacatacttttcttttacatgccagtttcatatatgatcttcttataggtttgattctcgaagaatgtttatttttaacactatgtaattctttcttaggccatatcctaaacaacttgctagtaaagtctaaaattttaatgatcaaacaattaataataaaattaaaaaagttattatgatctccccctatattaagtttagaatttcaaaaatatctaagtgacaattgagcaagtacacacagcataacacaatctaccaatatatcatactttattctagggtctacagctcctatacttaggtcaaatcttacttattgaaattagagacataacttatccattccttttgtactcataatatgccaagtcttatgcataaatttttatcataatgcttagtgagcttaaacctgagctttgaatctatttctactatgtacattacatccctagtgatcacaactttaagttcaaatatcaattaagttataatccctaataatcataacctagctctgacactactttgtcatatctcgatcccagcatctggattggccacatgatggccgtatactttctaggacaagatcctaaagaatatgcaagattttaaattcattacaatcttaaaatcccatgagtactattgatctgaattcaaaatgaatattacacattaacaaataccaaaccttgtataatttataaaatttgatcatctgattggtattgatgatattccatctaatcaattctttcaaccttgatcccacctatagtaaaatacatatatcctattactctgaaaatgaaaagaaagatgtgagcttcatagatcagtaagaattttcacacatcaatattaatataataataaataaaaaattatcaatagttattactcatataaatctcatacaataggatatcacgatcataaaatatatataaaaaaatatatttttttgtataataaatcacatcaaatacttttctcaatttatagtgtatcagatcctataatatttaggtgctaggctctaaattattacaactatattttccactcatggcatgacatcgacatagtggctaatctcgtggactatcacaatcacatttttagcatgaggttggacataagcatagtggctaatctagagggtcataagtgctcaactctgactaccacaatcatatttatagtccatattgggatatcaataaaatggctagttcagaaaactacaagtactcaactctaaactatcataaccattttctagcccataatgatgcatcaacaaaatagcaagcctagagcaccacaatccaccattcaaggacacaattcaattaataatttaagatcatatttctttgtaaaatttaatacataaaattaccaaaaccactcaagttgggataagtgacatgtgatatataactttatacagaatcatatatatacttaagaaataaatgtatagcatataactatcaaaaactatatagatcaaaatcattaattcacaaaaataatttttatataaatagatgattattatccagaaattcttatctctactaatgacaaactcggttacaactattttcttgtccatgcctaatatatccaaccaatcaacataattccaactcatccttaatcaaccattagcataaaaaataaataaattactcatagtgttttaatttcaaagctttaatatccaaattaatcttaaatctaatttttttgtactttctaatttaaaattatataattaaataagaaattaagattttatcttgacttgtaaactaaagcataacatttcttgctttgcattttcttattgggatgattgctctcatccaaaatcaaaaccaacaatcaaaaaaagctataaaatagttattgtcgatggtggaaaacttagaagaagaacagttggtcgaataataacatccgatggcccaagtgggtttgatctagatgccttagctagtgattcaagaatatgacaaagatcgtctatgatgggtttcgtggttacttgatcaagatcaaggtgaagaagggcaagattagtaggatccaatgaccagtgtcagcccatctaggtgatccaaatgattaatctataattaagagccaaattatagctcaatagaaatttggcgatgataaaatccaacaatgcccggcaagagtcgggttaggagtgaacaattatagagcaaacctagaatcttcttttgggatcaacctagggtctaccatcaagtcttctaaatctatctttctttcttttttttttatgcctaaatccaagaagagagataacaataaagggagaatgtagagagagatgtgagagagggaagaacaaatcgaatctctctctctcttccttgtcttctttcttcaaatagaagattttcctttctccctctcgtcctccaaacatggcagtggatggctgaggccaatagcctgcgatggcatccaacagtgaagaggcgacaaccccagtagcaacaatgatggcagctggtggcagcagtgggcaatcggatataaagaagaagaaaatcaaagcaaaataaggcaagaggccttgctttgatccaatgaagaggacttcaaggtaatcggtagcaatggtaagcatctgcaccagctcaacctttggtaatggtcgcaatggcaataatggccatggtggccaatggtggatgaatcaaagaaaataggggatccttggtgcggcaagatctaatggtctttcaagaagatcttagccaacaatggctacacgaggataggaaggaaggagagaaagtgcccatgagatgggaggcgatgcctcatctcctatgatggttcaaccacgattgggctaaaacttttttctcctccttttttgacaatgcaatcaattgatttctcaatagaatggaagctcagctttatagacaatggaggctaaggtttcctagcatccaatgagtttgagaagaagcaggagtcggactccttcgactcaaacaaaggaaaggaaggtctttccttccttcctattgtttcttgggcttgattttgggcctatttcgatgatgggctgggtagggtatcacaacagttttgtttctaaattgtcattatcaggaagagtaattctttgtacaccacattcgatgtagaaaaactgcaccactccgcctaattgagccacatataaccaccacttttcaatgtaatttagtattcgcaattctgctttttttttttgaaattttgaatggcaaaaatgtccatcctcttttaaaaaaatatgatatcttatggccatattataatatcctgcaatcaaattatgatttcttatatacagaaagttataatttgaccacaggatgtcataagaaaggggtggacattttcatcatttaaaaattttaaatttttaataatgaaaatgtcctttcctttttatgacttttggaagaaaaattatgacatcctatgtgtattaagacataatttgaccgcaaaatgtcataatttttttaaaaaaggataagcattttcatcatgcaaaatttcaaaagaaaaagcagaaatgcggatattaaatacacattggaaaacggtgcattacatgactcaatcagatggcgcggtgcattttttctacaccaagtgcggtgtacaaagaatttctcttactaggaaatatcttgagtctagaccggcccatttgtcatttagaccaatcaaggactatgaattttggtccataataagtatgagatcgtcatgggattgaaaaagatgggaaataactcctcagtttgccccttgacagctcacaattcttcaaataatagcataaatcattttttgaatcatcaaatttattacattttagccttttagaagaaaccaatgctatccatataaaaggtatttgttttctattaatgtcattgcactaatgaagacagcttcagcaaagatagagcagaaatcctttaaattttgtaagattcatttgatcatcttgaattttctttgatgatgtggttgcagcaatactatcagcaggaatcagcaaagttgcgccaccagatacagattttacaaaatgcaaacaggtgaacctcaaacttagatcagaactgattggtctcaaatacaatgtatatgcattttcaaagcttaagattatgtcttaccatgattcctaatctaccacctctacctttcaggcacttaatgggtgaagctttgagcactctgactgtaaaggagctcaagcaactcgaaaacagacttgaaagaggtatcacacggatcagatcgaagaaggtaatctgcatctatattttcttcaaactgagatcttcatattgccaccagcacatggcttatctgaagtacatgattattaatcatgaaacatcatgctatgcagcattgaaaagggaaatcattgtggttcacaggtgggggtagagcatgtaagatacgatgggatctaaaaatcgagtcaatataaataagtgtaatttgtattctgttctgcccccagaaatcagcataggcaccatgatgcatgtaccatcacctaataatatgcaacttcagaattttttggcccatccagctctttaatttgatttttgatgcatctcattgtttttttcgcatcagcatgagctgttgtttgcagagatcgagtatatgcagaaaagggtaatattctaaacttattccctgcaacttaattcaaagtattgatttctttcattcatgtctccctctgagtggttctttgttgttgaactgtaggaagtagaactccaaaatgacaatatgtacctcagagctaaggtatcaatgagaacaaaactctcttccttgtccttgtctgctatttctttctgatataaacaaaagaaatggatatcatattcgtaaaatatttgatatcatctatcatgcttttagacttatatgtggtactagcatggagccaaattatatgcattttcatatgtttagaatgcatgactaacgaaacagtgacttatgtttaaaatgcattttctcattgatcaaatttttttttacatactgttgaatttaacagaggagaatagtttccaagagatattacaaaacaagagtttatttgtatttgcttgtcttcaagaaatgaattcagctccactagtggtaatcatgtggtcatcatccatagtggcctgtatggcatggcataaaaactaggtgagattgtaaacaatcttcatgatgatagtatatatatcatagaacattgagcctttgtgtggaggctcatctgaaaattagtcatatctgaatgagaaccagattgatggaccgtttgaatcaagagataggacaagcaatactcgaaaaagtgccttagttacagcccaaattctggattgctgatttctctatttatcgatgcaccaacacccttcatgggcaagaatattgtttaaatcagtgttgcatttgacttcaaacctctaacatctcaacaaccataactgaagccccttcaaagctaaaatgcctgttaatttgttcttcacaaagaaaatggcattttttcctagatgtccataccgatactaacggtattttggaggcttgatgatgtgctaatgacactttggattcctcaaagaaatggctcctctgctccatctcggtcacaagtctctaaaattttcacttgttgtttccattgattctatttctttatattttatttagatcttcacagacacagtctcaaagtagcaaggtggcatctacattcttatttctcacttcaaaatttttggtgttctcagatagcagagaatgagcgagcacagcaagcaggtattgtgccggcagggcctgattttgatgctcttccaacgtttgataccagaaactattaccatgtcaatatgctggaggcagcacaacactattcacaccatcaagaccagacaacccttcatcttggatatgaaatgaaagctgatccagctgcaaaaatttactttaagtatgtcgctgcttgttaatgacatgttctaataacataggctaca

SEQ ID NO: 9 - DNA, 20 bp, Artificial Sequence
  synthetic PCR amplification 5′ forward primer for SHELL alleles
tcagcagacagaggtgaaag

SEQ ID NO: 10 - DNA, 20 bp, Artificial Sequence
  synthetic PCR amplification 3′ reverse primer for SHELL alleles
ccatttggatcagggataaa

SEQ ID NO: 11 - DNA, 49 bp, Artificial Sequence
  synthetic portion of Elaeis guineensis wild- type SHELL allele
gccgaaatggactgctgaagaaagcttatgagttgtctgtcctttgtga

SEQ ID NO: 12 - DNA, 49 bp, Artificial Sequence
  synthetic portion of Elaeis guineensis mutant MPOB-sh SHELL allele
gccgaaatggactgccgaagaaagcttatgagttgtctgtcctttgtga

SEQ ID NO: 13 - DNA, 49 bp, Artificial Sequence
  synthetic portion of Elaeis guineensis mutant AVROS-sh SHELL allele
gccgaaatggactgctgaagaatgcttatgagttgtctgtcctttgtga